(19) United States
(12) Patent Application Publication
(10) Pub. No.: US 2013/0131501 A1
(43) Pub. Date: May 23, 2013

(54) NEURO-VASCULATURE ACCESS SYSTEM AND DEVICE

(71) Applicants: Michael Blaivas, Cumming, GA (US); Timothy Chinowsky, Seattle, WA (US); Kurt Duclos, Kenmore, WA (US); John Zhang, Seattle, WA (US)

(72) Inventors: Michael Blaivas, Cumming, GA (US); Timothy Chinowsky, Seattle, WA (US); Kurt Duclos, Kenmore, WA (US); John Zhang, Seattle, WA (US)

(21) Appl. No.: 13/680,709

(22) Filed: Nov. 19, 2012

Related U.S. Application Data
(60) Provisional application No. 61/561,683, filed on Nov. 18, 2011.

(51) Int. Cl. A61B 8/14; A61B 8/00

(52) U.S. Cl. CPC A61B 8/14; A61B 8/4455; A61B 8/4461
USPC 600/424

(57) ABSTRACT

A neuro-vasculature access system and device for placing a cannula adjacent to a nerve. The neuro-vasculature access system and device includes a needle injector pivotally attached to an ultrasound transceiver which is operated to place a sterilizable needle or needle/cannula unit within the neuro-vasculature of a patient by a single user-device operator in which the neuro-vasculature is made visible in a monitor image by ultrasound insonification. A guidance template having an expected path trajectory is overlaid on at least one of a transverse short axis, a longitudinal long axis, or a three-dimensional image of the neuro-vasculature and predicts path of the needle or needle with overlapping cannula when it undergoes movement implemented by a controller located on the needle injector. The needle injector, ultrasound transceiver, and needle or needle/cannula unit may be contained within a flexible sheath capable of being sterilized.

### CROSS REFERENCES TO RELATED APPLICATIONS

This application claims the benefit of priority to and incorporates by reference in its entirety U.S. Provisional Patent Application No. 61/561,683 filed Nov. 18, 2011. This application also claims the benefit of priority to and incorporates by reference in its entirety U.S. patent application Ser. No. 12/986,143 filed Jan. 6, 2011 that in turn claims priority to U.S. Provisional Patent Application No. 61/293,004 filed Jan. 7, 2010. All patent applications incorporated by reference in their entirety.

### FIELD OF THE INVENTION

Disclosure herein is generally directed to the field of blood vessel and tissue access related devices, systems, and methods.

### BACKGROUND OF THE INVENTION

Medical personnel can be faced with patients who present arteries, veins, and nerves that are difficult to access with a needle and any needle-cannula assembly due to the qualities of the overlaying skin and/or the size and configuration of a given artery, vein, or nerve and the techniques undertaken to access a given structure within the patient's neuro-vasculature. The vein or artery may be obscured due to overlying fatty tissues or lack of sufficient blood flow may insufficiently fill the lumen to make the blood vessel palpable, as occurs with blown veins compromised with a hematoma, or veins that are otherwise structurally compromised as found in the elderly, intravenous administered drug users, and critically ill patients with very low blood pressure. Such patients as these, as well as with obese patients, prove difficult to cannulate under “blind” procedures. In many cases these patients have to endure multiple stabs with a needle, sometimes with penetration through the posterior wall of a vein before a successful placement of the needle is achieved and stable residence of the cannula or catheter within the blood vessel is achieved. Even allowing for an occasionally successful blind stick-and-insert catheter operation, the inserted catheter, if entered at too sharp an angle into a given blood vessel, may yet kink on insertion and thus hamper fluid delivery or removal into or from the blood vessel lumen. Moreover, current ultrasound image guided blood vessel access procedures require two people, one person to hold the ultrasound probe to secure an image to guide by, and another person to insert the needle/cannula. The prior art thus requires a minimum of three hands, a first person to hold the ultrasound transceiver and operate the ultrasound transceiver controls and nearby imaging systems, and a second person to work in tandem in close proximity with the first person to handle and insert the needle/cannula while observing the ultrasound image procured from the first person. With current blood-access ultrasound image guided devices, the first person commonly utilizes both hands and second person at least one hand to do the needle insertion, for a minimum of three handed, and thus a two-person operation. Accordingly, there is a need for solutions for difficult-to-access blood vessels and nerves that do not require two people to perform, and which are more precise than is offered by current devices and procedures.

### BRIEF DESCRIPTION OF THE DRAWINGS

Preferred and alternative examples of the present invention are described in detail below with reference to the following drawings depicted in FIGS. 1-45:

FIG. 1 schematically depicts a blood vessel access handset 10 that images blood vessels utilizing B-mode based single scan planes and rotationally-configured scan plane arrays;

FIG. 2 schematically depicts the handset device of FIG. 1 equipped with an detachable attachable needle injector and cannula placement cartridge;

FIG. 3 schematically depicts the handset 10 with attached cartridge 90 during a blood vessel survey and cannula placement operation on the peripheral vasculature of the patient's arm;

FIG. 4 schematically depicts an embodiment of a blood vessel access system 200 including the handset 10 of FIG. 1 deployed from a movable cart;

FIG. 5 schematically depicts a bottom perspective view of the handset 10 depicted in FIGS. 1-3, without the cartridge 90;

FIG. 6 schematically depicts components within transducer housing 12;

FIG. 7 schematically depicts a cutaway perspective view of the friction hinge 38 connecting between injector arm 40 and transducer base 16 depicted in FIGS. 1, 5, and 6;

FIG. 8 schematically depicts another cutaway view of the friction hinge 41 connecting between injector arm 40 and transducer base 16;

FIG. 9 schematically depicts a perspective view of injector arm 40;

FIG. 10 schematically depicts a cutaway perspective view of the injector arm 40;

FIG. 11 schematically depicts a close-up cutaway view of the component parts holding or interacting with friction hinge 41;

FIG. 12 schematically depicts friction hinge 41 removed from its holder;

FIG. 13 schematically depicts a perspective cross-sectional view of the friction hinge region 38 spanning between the injector arm 40 and transducer base 16;

FIG. 14 schematically depicts a perspective view of the bottom region of the handset 10 illustrating the transducer 135 emanating a scan plane 175 substantially perpendicular to the needle 120 that crosses through it;

FIG. 15 schematically depicts a perspective view of the scan plane 175 intersecting across the short axis of blood vessel BV that shows the needle 120 poised to enter the anterior wall near the midline of the blood vessel BV;

FIG. 16 schematically depicts a perspective view of the bottom region of the handset 10 illustrating the transducer 135 emanating a scan plane 175 substantially parallel to the needle 120 that transits;

FIG. 17 schematically depicts a perspective view of the scan plane 175 intersecting the across the long axis of blood vessel BV that shows the needle 120 poised to enter the anterior wall near the midline of the blood vessel BV;

FIG. 18 schematically depicts the touch screen monitor 206 presenting a home screen illustrating a panel of four blood vessel based access procedures;

FIG. 19 schematically depicts the handset 10 surveying for a peripheral vein undertaken during the IV procedure selected from the home screen depicted in FIG. 18;

FIG. 20 schematically depicts an ultrasound image presented on the monitor 206 while surveying for a vein undertaken during short axis mode when the scan plane 25 emanating from the transducer 20 intersects blood vessels substantially at a perpendicular orientation;

FIGS. 21 and 22 schematically depict the differential collapsibility or compressibility of veins and arteries when subjected to pressure of the transducer base 16 exerted onto the patient's arm;

FIGS. 23 and 24 schematically depict the differential compressibility of veins and arteries as presented on screen images on touch screen 206;

FIG. 25 schematically depicts an ultrasound image of a long axis view of a targeted blood vessel presented on a monitor 206;

FIGS. 26 and 27 schematically depict the attachment of cannula cartridge 90 to injector arm 40;

FIGS. 28A-29B schematically illustrate the “cannulate” step represented in access menu 280 and sets forth how the controller's 47 push and toggle buttons 42, 44, and 46 are used in a needle injection and cannulation procedures employing the cassette 90 that is mounted to the slot 54 side of the injector arm 40;

FIG. 30 schematically depicts a screen image of penetration of the needle 120 with overlapping cannula 140 into a blood vessel near midline when the injector arm 40 is approximately at a 30 degree angle relative to the base of the transducer 135;

FIG. 31 schematically depicts a screen image after penetration of the needle 120 for advancing the overlapping cannula 140 into the blood vessel when the injector arm 40 is approximately at a 20 degree angle relative to the base of the transducer 135;

FIG. 32 schematically depicts a screen image after cannulation of the blood vessel;

FIG. 33 schematically depicts a touch screen selection of an arterial access procedure activated on the home screen;

FIGS. 34-36 schematically depict a detachably attachable sterile transducer cap;

FIGS. 37-39 schematically depict the covering of the handset 10 with a sterile sheath 300 and attachment the cartridge 90;

FIG. 40 depicts a perspective view of the cartridge 90;

FIG. 41 schematically depicts a partial cut-away and perspective view of the cartridge 90;

FIG. 42 schematically depicts a close-up of an ejector bar 104 that pushes open guide doors 105;

FIG. 43 schematically depicts the retraction of needle mount 92 from cannula mount 98 and cannula mount's 92 release of cannula 140;

FIG. 44 schematically depicts removal of the handset 10 with opened guide doors 105 leaving cannula 140 placed in the patient's arm; and

FIG. 45 schematically illustrates placement of the needle 120 and overlapping cannula 140 adjacent to a nerve within an insonified region of the patient's neuro-vasculature.

### DETAILED DESCRIPTION OF THE INVENTION

The invention generally concerns a neuro-vasculature access system and device for placing a cannula adjacent to a nerve, or alternatively, within a blood vessel, leaving the external portion of the cannula protruding from the patient's skin for either subsequent fluid communication with the nerve or the cannulated blood vessel. As regards accessing a nerve, the neuro-vasculature access system and device includes a needle injector pivotally attached to an ultrasound transceiver is operated to place a sterilizable needle or needle/cannula unit within the neuro-vasculature of a patient by a single user-device operator in which the neuro-vasculature is made visible in a monitor image by ultrasound insonification. A guidance template having an expected path trajectory is overlapped on at least one of a transverse short axis, a longitudinal long axis, or a three-dimensional image of the neuro-vasculature that illustrates the predicted path of the needle or needle with overlapping cannula when it undergoes movement implemented by a controller located on the needle injector. In alternate embodiments the needle injector, ultrasound transceiver, and needle or needle/cannula unit may be contained within a flexible sheath that is capable of being sterilized.

As regards accessing blood vessels, embodiments of the invention concern a single-person operable device configured for projecting ultrasound energy into a patient and generating acquired ultrasound based images for the purposes of selecting a blood vessel for cannulation and to implement the cannulation of the selected blood vessel by the single-person operable device. The single-person operable device allows the single person to guide a needle and a catheter or cannula under precise mechanical control and place the catheter, also known as a cannula, reliably into the patient's vascular structure, in particular a targeted blood vessel selected by the person operating the device. The device is configured to allow the single person user-operator to acquire ultrasound images used for ultrasound image-guided blood vessel access procedures and to implement needle and catheter/cannula placement within the imaged, targeted blood vessel with either the device user's single hand or two hands.

The particular embodiments include an access system operated by a user to cannulate a blood vessel of a patient. The access system includes a handset having an ultrasound transceiver occupying a swivelable housing that allows left-handed or right-handed holding of the ultrasound transceiver against the surface of a patient. The ultrasound transceiver is equipped with a rotatable ultrasound transducer that is in communication with a computer processing unit. The rotatable ultrasound transducer is configured to insonify a region of the patient's vasculature beneath the patient's surface with B-mode ultrasound energy. Rotatable views of the insonified region are generated from signals relating to the echoes of fundamental and/or harmonic frequencies that are processed according to microprocessor executable instructions accessible by the computer processing unit. By rotatable views it is understood that a series of views may be generated in which each view within the series has a different perspective of the patient's vasculature from the preceding view depending on the change in angular rotation or angular increment that is undertaken by the rotatable ultrasound transducer between rotatable views. The access system further includes a needle injector that is pivotally attached to the ultrasound transceiver and configured to convey positional information relative to the rotatable ultrasound transducer to the central processing unit. Another term for the needle injector is injector arm. The needle injector or injector arm is further configured for detachable and slideable connection with a needle and a cannula, the needle being configured for slideable connection within the lumen of the cannula. The needle injector or injector arm further includes a controller configured with single-function pushbuttons and a multiple-function toggle button that is operable by the user to change the position of the needle, the cannula, and the rotatable ultrasound transducer. Other components of the access system include a monitor configured to present images of the rotatable views. The images having an orientation selected by the user to undertake penetration of the blood vessel within the insonified region by the needle and the cannula substantially along a trajectory overlayable on the images based on the positional information determined by the central processing unit. The monitor may include touch sensitive surfaces.

The access system is further characterized in that the slideable connection of the needle and the cannula may be motorized within the needle injector. Furthermore, the rotatable views include a substantially short axis cross-sectional view and a substantially long axis cross-sectional view of the blood vessel within the insonified region. The angular change undertaken by the rotatable ultrasound transducer between the short axis and long axis cross-sectional views may be less than ninety degrees, substantially ninety degrees, or greater than ninety degrees. In general terms the substantially short axis view of the blood vessel is employed by the access system user to align the needle to penetrate near the midline of the targeted blood vessel, and the substantially long axis view of the blood vessel is employed by the user to visualize the advancement of the cannula further into the blood vessel lumen and/or to visualize the retraction of the needle from the blood vessel and nearby visible tissues. Oftentimes the short axis cross-sectional view is used to penetrate the targeted blood vessel at a more acute angle to the targeted blood vessel than the following cannulation procedure when the cannula is advanced more forwardly or deeper within the lumen. To provide the maximum flexibility of movement while preserving a minimum of induced vibration, the controller is configured with push buttons, some of the push buttons having multiple functions to allow the efficient movement of the needle towards or away from the blood vessel along with the cannula, the needle towards or away from the cannula, the cannula towards or away from the needle, and changing of rotation to get different rotational views of patient's vasculature within the insonified region of the rotatable transducer. The rotatable transducer is motorized to effect its rotation. The angular rotation of the rotatable ultrasound transducer may be in increments of ninety degrees, or may be varied in increments less than 90 degrees or increments greater than ninety degrees. Thus the aforementioned rotatable views may have a change in perspective of the patient's vasculature of ninety degrees, less than ninety degrees, or greater than ninety degrees depending on the change in angular rotation undertaken by the rotatable ultrasound transducer.

The access system is further characterized in that the pivotally attached needle injector includes a friction hinge that allows the injector to be set to and remain at a given angular position relative to the targeted blood vessel or the transceiver housing or the rotatable transducer. The friction hinge includes position sensors that are configured to provide angular information of the friction hinge for determination of the trajectory within the insonified region that the needle or needle with overlapping cannula may follow within the insonified region. The friction hinge is also configured to provide any change of angular or positional information that is undertaken by a change in the friction hinge position for re-determination of a change in trajectory of the needle and/or the needle with overlapped cannula is expected to follow within the insonified region.

Other characterizations provide for the controller occupying the injector arm to advance synchronously the needle with the cannula in the direction towards the targeted blood vessel or away from the blood vessel. Moreover, the controller may be configured to advance or retract the needle independently of the cannula, to advance or retract the cannula independently of the needle, and to change the rotational views of the insonified region whereupon the change in rotational views include a back and forth representation of real time or lively acquired ultrasound images of the insonified region having either a substantially short axis cross-sectional view or a substantially long axis cross-sectional view of the targeted blood vessel.

In another embodiment of the access system, the access system includes an ultrasound transceiver configured to be swiveled, pivoted, or turned to accommodate holding by left-handed or right-handed holding users, the ultrasound transceiver having a rotatable ultrasound transducer in communication with a computer processing unit, the ultrasound transceiver handheld by the user against the patient to obtain rotatable views of an insonified region, utilizing B-mode ultrasound, of the patient's vasculature relating to the signals of ultrasound echoes processed according to instructions executable by the computer processing unit. The access system further includes a needle injector having motorized platforms and a controller operable by the user to change the position of the motorized platforms and the rotatable transducer, wherein the motorized platforms include a first slideable mount and a second slideable mount, and the needle injector being pivotally attached to the ultrasound transceiver and further configured to convey positional information relative to the ultrasound transceiver to the central processing unit. In this alternate embodiment, the access system is equipped with a cassette that is configured for detachable connection with the needle injector such that the cassette includes a needle that is detachably attachable with the first slideable mount and a cannula that is detachably attachable with the second slideable mount, wherein the needle has a slideable connection within the lumen of the cannula and may be disconnected or slid out of the lumen of the cannula.

Other embodiments provide for a vascular access system for cannulating a blood vessel of a patient. The access system includes a handheld ultrasound transceiver having a rotatable ultrasound transducer in communication with a computer processing unit. The ultrasound transceiver is configured to generate rotatable views of an insonified region of the patient's vasculature. The access system further includes a needle injector that is pivotally attached to the ultrasound transceiver and configured to convey positional information relative to the rotatable transducer to the central processing unit. The needle injector includes a controller that is operable by the user and is configured to advance the needle towards the patient and/or change the rotational position of the rotatable transducer. The system further includes a monitor configured to present images of the rotatable views. In yet other embodiments, the vascular system includes a cannula that is in slideable connection with the needle, and the central processing unit is configured to generate positional information in an image overlay having at least one vertical axis associated with the position of the rotatable transducer and at least one horizontal axis associated with the needle injector, the intersection of the horizontal and vertical axes providing a sighting aid for needle and cannula placement within a targeted blood vessel. The overlay further provides a predicted pathway that the needle and/or cannula will follow during transit through the insonified region. In yet other embodiments the overlay may include an icon indicative of the rotational status of the rotatable transducer in which the icon can change appearances to indicate that a particular rotational view being presented on the monitor is a short-axis cross-sectional view or a long-axis cross-sectional view of the blood vessel targeted for injection and/or cannulation. Other embodiments of the access system provide for the controller of the needle injector to move the needle towards or away from the blood vessel independently of the position of the cannula, to move the cannula towards or away from the blood vessel independently of the position of the needle, or to synchronously move the needle and cannula together towards or away from the blood vessel.

Another embodiment provides for a blood vessel access system operable by a user having an ultrasound transceiver configured for left-handed or right-handed holding by the user, the ultrasound transceiver having a rotatable ultrasound transducer that is in signal communication with a computer processing unit and is configured to produce an insonified region of the patient's vasculature while the ultrasound transceiver is handheld against the patient. Rotatable views of the insonified region relating to the signals of ultrasound echoes processed according to instructions executable by the computer processing unit and displayed on a monitor in signal communication with the central processing unit that is viewable by the user operating the ultrasound transceiver. Attached pivotally to the ultrasound transceiver is a needle injector having at least one motorized platform having a needle and a controller operable by the user to rotate the rotatable transducer and to change the position of at least one moveable platform. The access system further includes a cartridge having at least one slideable mount having a needle, the cartridge configured for detachable connection with the needle injector and the at least one slideable mount configured for detachable connection with the injector arm's at least one moveable platform. In response to signals conveyed from the controller operated by the user to the at least one moveable platform in removable connection with the at least one slideable mount, the needle is moved from the cassette to penetrate the patient and be visible within the insonified region shown in the rotatable views presented on the monitor.

In other alternative embodiments of the blood vessel access system above, the system's cartridge may include a cannula configured for detachable connection with the at least one slideable mount and in slideable communication with the cartridge and the needle, and controllable by the controller to at least move within the cartridge, from the cartridge to the insonified region, and within the insonified region. The images presented on the monitor are viewable and adjustable by the user operating the controller to obtain an orientation selected by the user to undertake penetration of a targeted blood vessel by the needle and the cannula within the insonified region. The access system further includes an overlay generated from positional information by the central processing unit that displays at least one of a vertical axis to denote or indicate the position of the rotatable transducer within the insonified region, a horizontal axis to denote or indicate the position of the injector arm relative to the rotatable transducer, and visual representations indicating a trajectory or pathway traversable by the needle and the cannula within the insonified region. The positional information is determined from microprocessor executable instructions applied by the central processing unit to signals conveyed from position sensors located in the injector arm and the motorized rotatable transducer. Changes in the trajectory overlaid onto the images are determined from changes in angular information caused by changes in the needle injector's position relative to the rotatable ultrasound transducer. Further alternate embodiments provide for the needle injector to include a friction hinge configured to maintain the needle injector at an angular position selected by the user to cannulate the blood vessel along the trajectory presented in the overlay, and any changes to the position of the friction hinge as a consequence of the user changing the position of the injector arm is detected by position sensors that generate signals processible by the computer processing unit.

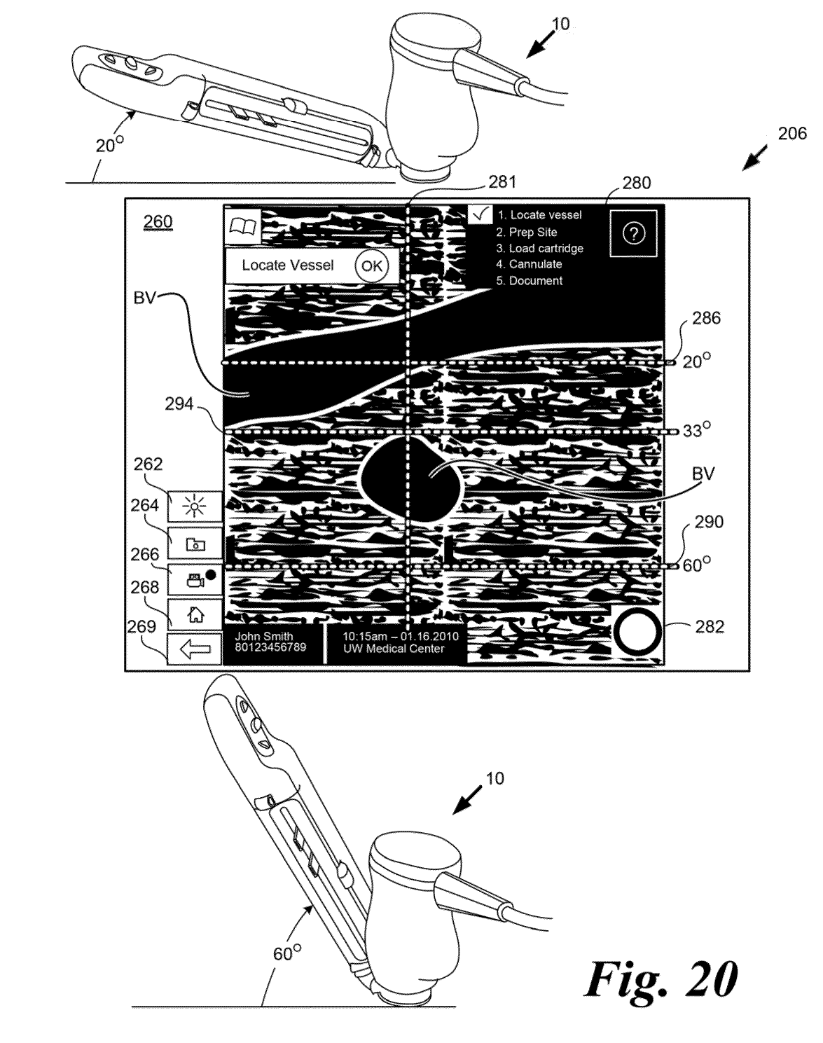

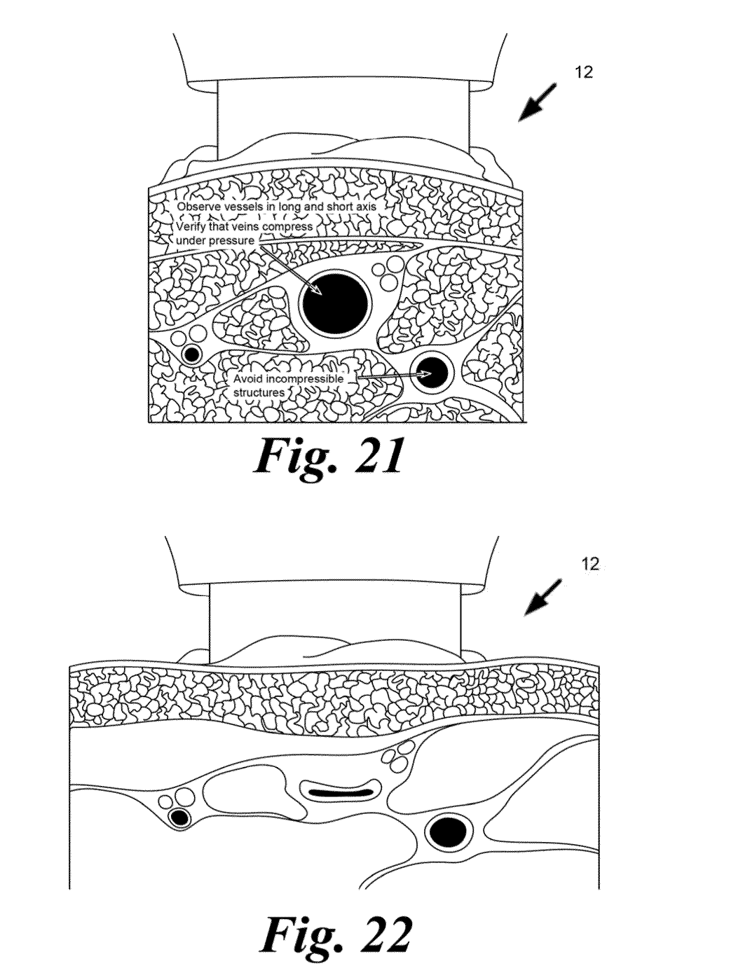

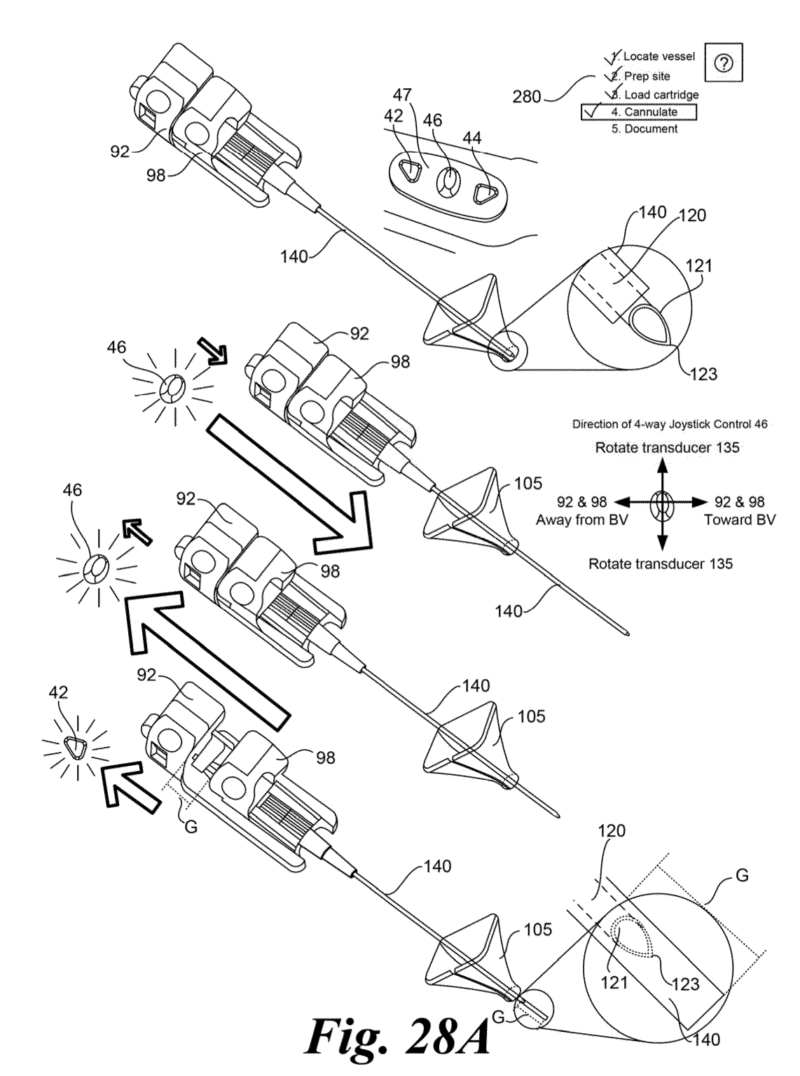

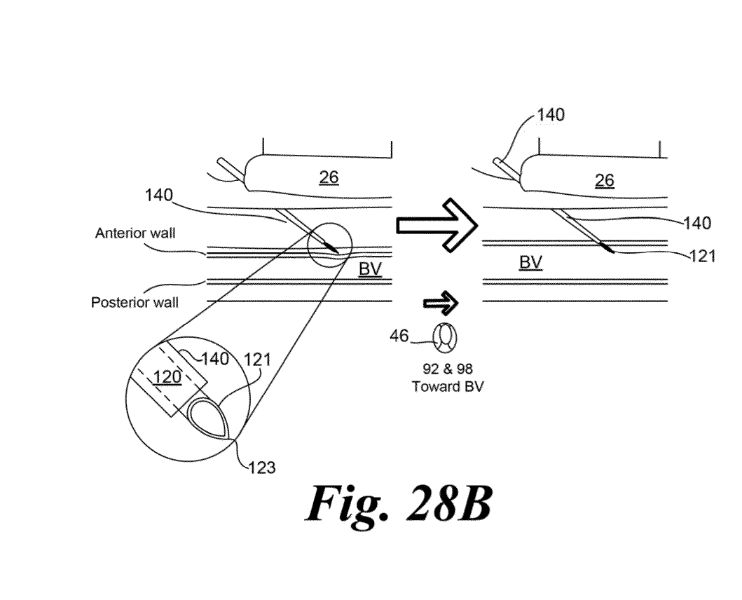

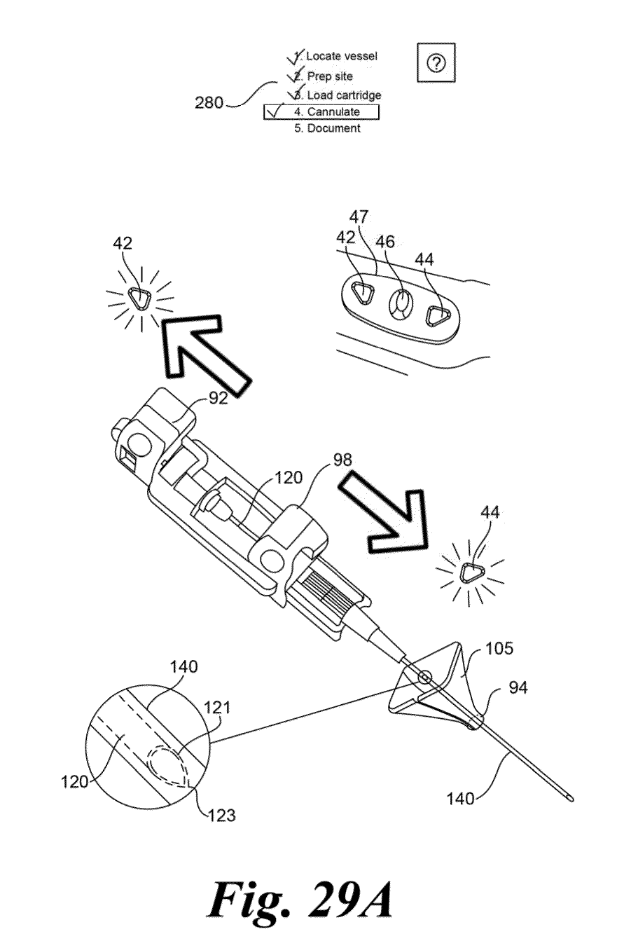

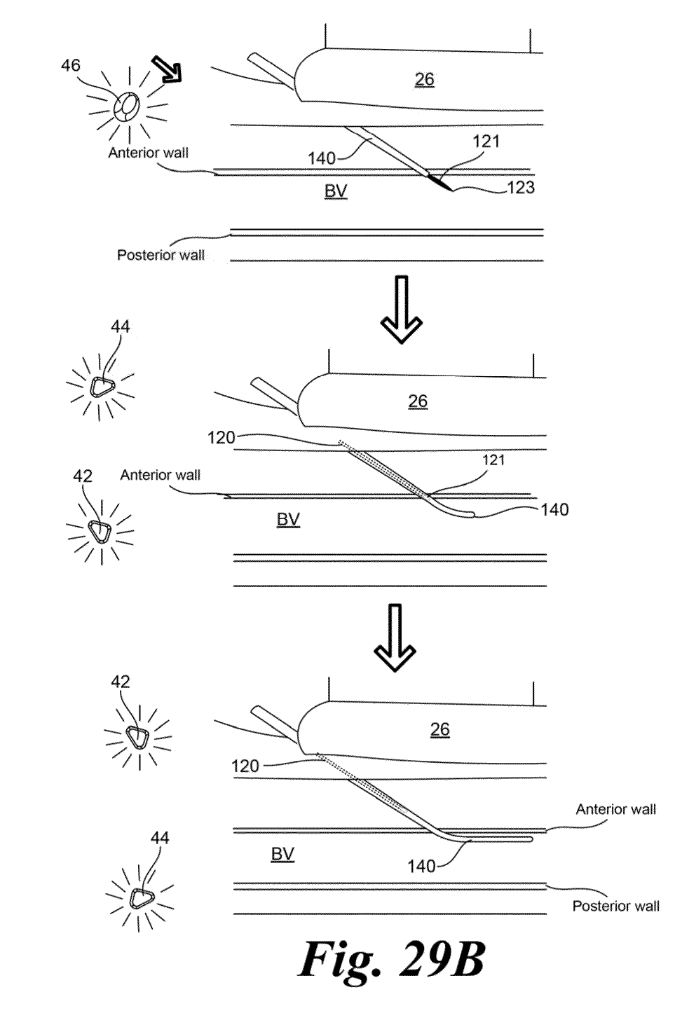

In yet other alternative embodiments of the blood vessel access system above, the system's motorized platforms include a first slideable mount and a second slideable mount, the first slideable mount in detachable connection with the needle and the second slideable mount in detachable connection with the cannula. The controller is further configured to advance the first slideable mount synchronously with the second slideable mount towards or away from the patient's vasculature, and/or to advance the second slideable mount towards or away from the patient's vasculature independently of the position of the first slideable mount. The controller thus can move the needle and the cannula together at the same time at the same rate, either towards the patient's vasculature or within the patient's vasculature and the insonified region of the patient's vasculature. The controller can also move the needle separate from the cannula, or the cannula separate from the needle, to in effect create user-selected gaps between the first and second slideable mounts operating within the cartridge to causes gaps in the distal ends of the needle and cannula. Thereafter, at the discretion of the user viewing the rotatable views to accommodate needle penetration and cannulation of a targeted blood vessel, the needle and cannula may be synchronously advanced or retracted together with preservation of the user-selected gaps, or alternatively, change the gap distance between the slideable mounts and between the terminal ends of the needle and cannula by independently changing the gap sizes by selectively changing the position of the first slideable mount relative to the second slideable mount, and/or changing the position of the second slideable mount relative to the first slideable mount. Examples of synchronous and independent movement of the needle and/or cannula, with or without gaps created between the cartridge's slideable mounts are shown in and described in FIGS. 28A-29B below. In other embodiments the overlay applied to a particular rotatable view being seen by the user may include a position icon indicative of either a substantially short-axis cross-sectional view of the blood vessel being targeted for needle penetration and cannulation, or a substantially long-axis cross-sectional view of the targeted blood vessel. The position icon may be, for example, a circle for a short-axis cross-sectional view or a tube for a long-axis cross-sectional view. Other embodiments may provide that the trajectory overlaid on the images may resemble cross-hairs to function as a sighting aid that is formed from the intersection of the long axis and the horizontal axis when the rotatable views are presented as a short-axis cross-sectional view, or as an angled, substantially linear line when the rotatable views are presented as a long-axis cross-sectional view. Examples of components of the overlay for guiding the penetration of a needle with cannula into a targeted blood vessel and the subsequent cannulation of the blood vessel and retraction of the needle are shown in and described in FIGS. 20, 23, 24, 25, and 30-32 below.

The aforementioned embodiments further include a monitor configured to present images of the rotatable views, in which the images are viewable and adjustable by the user operating the controller to obtain an orientation selected by the user to undertake penetration of the blood vessel by the needle and the cannula near a trajectory overlayable on the images based on the positional information determined by the central processing unit. The monitor may include touch sensitive surfaces. These alternate embodiments provides for the controller to be configured to advance the first slideable mount with the second slideable mount towards or away from the blood vessel, to advance the first slideable mount with the second slideable mount from a starting locus of the second slideable mount wherein the cutting edge of the needle extends beyond the terminal end of the cannula that is designed for occupation within the lumen of the targeted blood vessel. The starting position serves to establish that the needle and the cannula, each respectively detachably attached to the first and second slideable mounts, may be a structure that functions as an engageable catch that temporarily holds the second slideable mount to the starting locus, thereby establishing a home or starting position from which the injector arm mounted cassette begins movement operations of the slideable mounts. The structure or cannula catch mount is detachably engageable so that the catch's holding forces may be overcome with enough motorized forces conveyed to the first and/or second slideable mount to commence needle puncturing and cannula placement procedures within the lumen of the targeted blood vessel.

In this and other embodiments of the aforementioned access system, the controller is configured to retract or advance the first slideable mount independently of the second slideable mount and/or the second slideable mount independently of the first slideable mount. Similarly, the pivotally attached needle injector includes a friction hinge having position sensors configured to provide angular information of the friction hinge for determination of the trajectory to be undertaken as the pathway the needle and/or the needle with overlapping cannula will follow near within the insonified region. Any angular change conveyed to the pivotable injector is conveyed by the signals from the friction hinged based position sensors to allow re-determination of a change in trajectory pathway that the needle and/or needle overlapping cannula will nearly undergo within the insonified region.

In yet another alternate embodiment of the blood access system includes the ultrasound transceiver configured for left-handed or right-handed holding, the ultrasound transceiver having a rotatable ultrasound transducer utilizing B-mode ultrasound. The rotatable ultrasound transducer is in communication with a computer processing unit, the ultrasound transceiver handheld by the user against the patient to obtain rotatable views of an insonified region of the patient's vasculature relating to the signals of fundamental and/or harmonic ultrasound echoes processed according to instructions executable by the computer processing unit. The blood vessel access system further includes a needle injector or injector arm having motorized platforms and a controller operable by the user to change the position of the motorized platforms and the rotatable transducer, the motorized platforms including a first slideable mount and a second slideable mount, the needle injector being pivotally attached to the ultrasound transceiver and further configured to convey positional information relative to the ultrasound transceiver to the central processing unit. Attached to the needle injector is a cassette or cartridge configured for detachable connection with the needle injector, the cassette having a needle detachably attachable with the first slideable mount and a cannula detachably attachable with the second slideable mount. The needle is configured to have slideable connection within and disconnection from the lumen of the cannula. The injector also includes a cannula release and a needle catch to hold the needle within the cartridge upon completion of a cannulation procedure. The access system also includes a monitor configured to present images of the rotatable views, such that the images are viewable and adjustable by the user operating the controller to obtain an orientation selected by the user to engage in needle injection and cannulation procedures. The system also provides for projecting onto the images an overlay having a predicted trajectory based upon rotatable transducer orientation to the blood vessel and the injector arm's orientation to the rotatable transducer. The overlay provides for the predicted trajectory to serve as the pathway the needle and/or cannula will undergo while transiting to and penetrating the blood vessel. The needle and the cannula transit along the ovelayable trajectory based on positional information of the rotatable transducer and injector arm with relation to the insonified blood vessels made visible on the images presented on the monitor. The positional information is determined by the central processing unit, and is used by the user to do at least one of advancing the needle into the blood vessel, retracting the needle from the blood vessel, advancing the cannula into the blood vessel, and retracting the cannula within or from the blood vessel secure. In other alternate embodiments the cartridge includes a needle catch configured to engage the cannula release so that the exterior portion of the cannula resides outside the patient's skin while keeping the interior end of the cannula residing within the blood vessel.

Similarly with the other embodiments described above, this alternate embodiment of the access system provides for the controller to be configured to obtain rotatable views that include a substantially short axis cross-sectional view and a substantially long axis cross-sectional view of the blood vessel within the insonified region to be used in selecting a pierceable locus for the targeted vessel (near the vessel's midline) for penetration of the needle (short axis) or to visualize the cannulation and needle withdrawal from the vessel's lumen (long axis) that are viewable within the insonified region presented on the monitor's screen. The controller is similarly configured to either move the first slideable mount with the second slideable mount towards or from the blood vessel, to move the first slideable mount towards or away from the second slideable mount and the second slideable mount towards or away from the first slideable mount, and to obtain with back-and-forth ease short and long axis cross-sectional views by the back-and-forth rotation of the rotatable transducer substantially at right angles or ninety degrees between rotations. This embodiment also provides that the pivotally attached needle injector is equipped with a friction hinge so that a particular injection or cannulation angle may be established during the motorized operations of the injector's moveable platforms. The friction hinge having position sensors configured to provide angular information for determination of the trajectory for piercing the blood vessel by the needle within the insonified region, or a change in angular information from a change in acute angle, say a lowering of the angle to a less-acute value that is more amenable to cannulation after penetration of the targeted blood vessel by the needle. The change in injector-to-blood vessel or injector-to-transducer values is conveyed to the central processing unit wherein a residing microprocessor utilizes the executable instructions to re-draw a trajectory pathway overlay onto the monitor presented images having the insonified region and adjacent borders that the needle and/or cannula will nearly follow to effect retraction of the needle from the vessel's lumen and forwardly sliding the cannula further into the vessel's lumen.

In this alternate embodiment of the access system, however, further defines the cannula release mentioned above to a pair of doors having an orifice sized to allow the passing of the cannula overlapping needle without substantial sideway slippage while engaging the blood vessel. Upon satisfactorily placing the distal portion of cannula within the lumen of the blood vessel and removing the needle from the patient's blood vessel and overlying dermus, the cannula release causes the doors to swing open. The swung open doors creates a larger space sufficient to allow the cassette to be removed from the external portion of the cannula emanating above the patient's skin without displacing the internal portion of the cannula residing within the blood vessel. This alternate embodiment of the access system also provides for the rotatable transducer to be covered by a sterilized cap for undertaking blood access procedures requiring an aseptic arena. For blood access procedures requiring a sterile arena, the transceiver body and adjoining injector arm may be overlapped by a flexible sterile sheath. The flexible sterile sheath includes fittings engageable with the motorized platforms of the injector and the first and second slideable mounts of the sterilized cassette, and may include flexible pleated folds to accommodate the displacement distances between the fittings attached to the motorized platforms that slide back and forth during blood vessel access procedures.

In greater detail, these embodiments relate to blood vessel access systems, devices, and methods for placing a needle within the lumen of at least one blood vessel. The blood vessel access devices aid the user in insertion of peripheral intravenous (IV) lines, central, and peripherally inserted central catheter PICC lines by improving both the visualization of the vasculature and manipulation of the needle. A compact ultrasound probe located in a transceiver handset provides real-time B-mode images of the anatomy to be cannulated. A motorized mechanism contained in an injector arm attached to the probe advances the needle and catheter into the ultrasound visualized blood vessel under local control from the user. As regards systems, disclosure illustrated and discussed below are drawn to an ultrasound transceiver that is sonically coupled to convey ultrasound energy into a patient, and to generate signals from received returning ultrasound echoes derived of fundamental and/or harmonic ultrasound energies to generate at least one image of the patient's sonicated region on a monitor in which the at least one image includes a single or multiple blood vessels ultrasonically made visible within the real time image. The system further includes a needle injector that is pivotally attached or connected with the ultrasound transceiver. The needle may be attached to an overlapping cannula, and the needle and/or overlapping cannula may be contained within a sterilizable housing that is detachably connectable with the needle injector. The needle injector is connected with a push-button and toggle based controller that controls the advancement or retraction of the needle from the sterilizable housing and rotation of the rotatable transducer. The system further includes software or executable programs having instructions configured to develop and overlay at least one aiming template or guidance template having needle/cannula predicted trajectories for a given angle at which the injector arm is held by the friction hinge. The aiming or guidance overlay includes a predicted path that the needle will undertake to reach and penetrate the lumen of the at least one blood vessel. The guidance overlay includes the predicted path to be undertaken on at least one of a transverse or lateral cross-sectional view, a longitudinal cross-sectional view, and a three dimensional view of the at least one blood vessel presentable within the at least one image.

Other embodiments provide for the access to peripheral blood vessel, for example veins or arteries, that are located approximately 3.5 mm to 35 mm beneath the patient's skin, or to access nerves occupying similar depths. The ultrasound-guided needle insertion and cannulation placement device is designed to make for example, the intravenous (IV) placement of cannulas into peripheral blood vessels, faster, safer, and less traumatic for the patient. Thus patients presenting challenging peripheral vascular anatomies, for example long term IV drug users, excessively obese patients, the elderly, or critically ill patients having low blood pressure will be safely and efficiently cannulated by the image-guided and precisely controlled mechanical features of the blood access vessel device and system.

In yet other embodiments the blood vessel access system, including the ultrasound transceiver, the injector, and any detachable needle/cannula housing units, may be enveloped within a flexible sheath that is capable of being sterilized. Sonic coupling gel may be applied between the transceiver and the internal surfaces of the flexible sheath, and between the patient and the external surface of the flexible sheath.

As regards to an access device for purposes of executing the image guided placement of a needle within at least one blood vessel, the access device includes pivotally connecting the access device to an ultrasound system. The ultrasound system includes a monitor and may be portable to assist in obtaining images of blood vessels beneath the neck, chest, abdomen, arms, legs, and other part of the torso that is ultrasonically visualizable. As with the access system, the access device includes software or executable programs configured to develop and overlay aiming or guidance templates of predicted needle pathways onto at least one of a transverse cross-sectional view, a longitudinal cross-sectional view, and a three dimensional view of the at least one blood vessel presentable within the at least one image.

Similarly in other embodiments, the access device and pivotally connected ultrasound transceiver, including any detachable needle/cannula housing units, may be enveloped within a flexible sheath that is capable of being sterilized. Sonic coupling gel may be applied between the transceiver and the internal surfaces of the flexible sheath, and between the patient and the external surface of the flexible sheath.

As regards methods of using an access device or access system, the method encompasses connecting a needle injector pivotally with an ultrasound transceiver having a monitor configured to present an image of at least one blood vessel, installing a sterilizable housing containing the needle and cannula, and operating the needle injector controller to place the needle within the lumen of at least one blood vessel presented on the monitor to which is overlaid a guidance template.

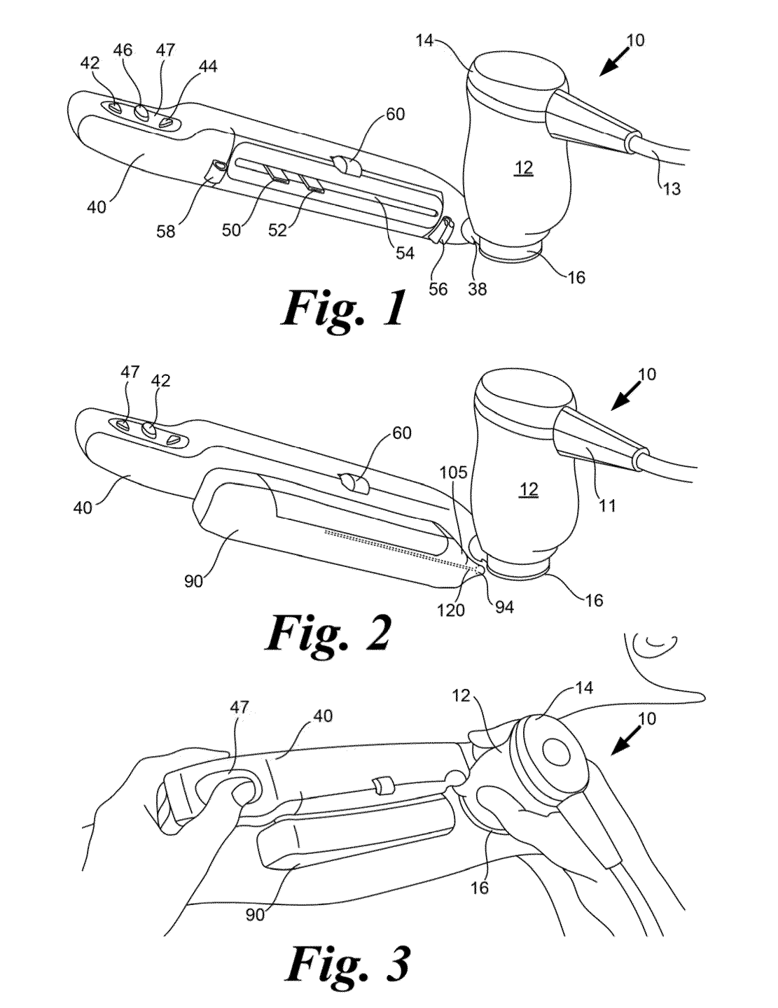

Different embodiments of the nerve and blood vessel access devices, systems, and method of using these devices and systems are described in FIGS. 1-45 below. The devices, systems, and methods may be employed to access nerves or to target blood vessels to allow hospital or clinic based personnel to undertake successful ultrasound-guided placement of needle or cannulas adjacent to nerves or to perform blood vessel access procedures. The blood vessel access procedures include short peripheral intravenous procedures (IVs), generally under aseptic conditions, and peripherally inserted central catheter (PICC) lines, and any difficult medical procedure currently using blind needle placement, generally under sterile conditions. Difficult medical procedures include nerve blocks, Thoracentesis and Paracentesis procedures, and biopsy procedures. Needles utilized by the devices and systems commonly cover 22 to 16 gauge needles and with the appropriate larger sized cannula or catheters that may be slideable over the 22 to 16 gauge needles. The needles and corresponding cannulas may be larger or smaller than the 22 to 16 gauge sizes.

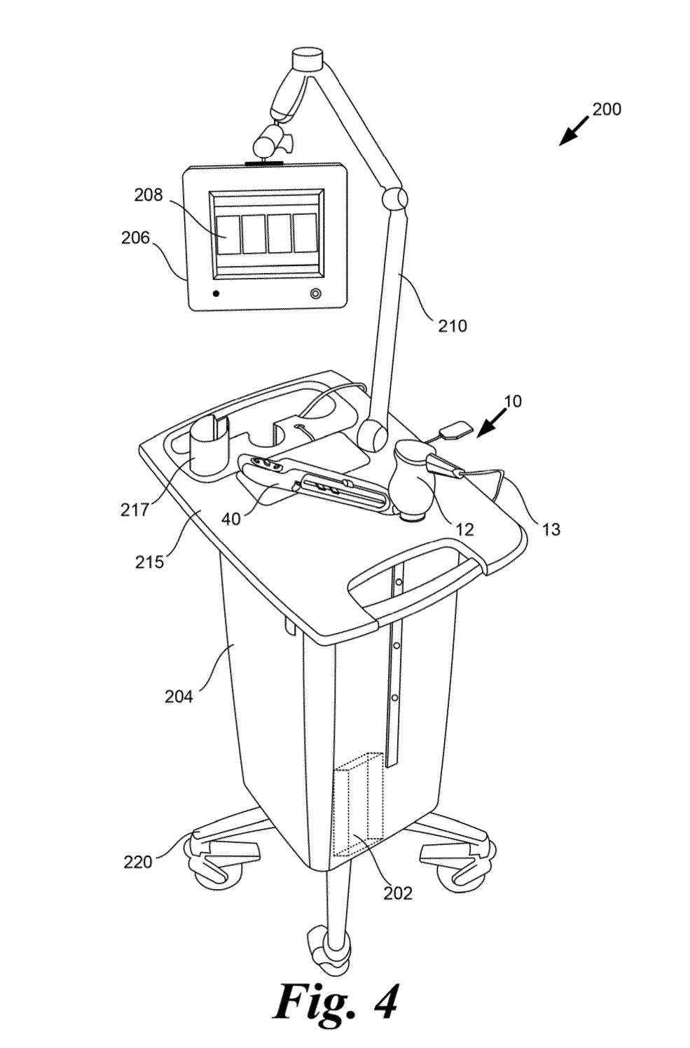

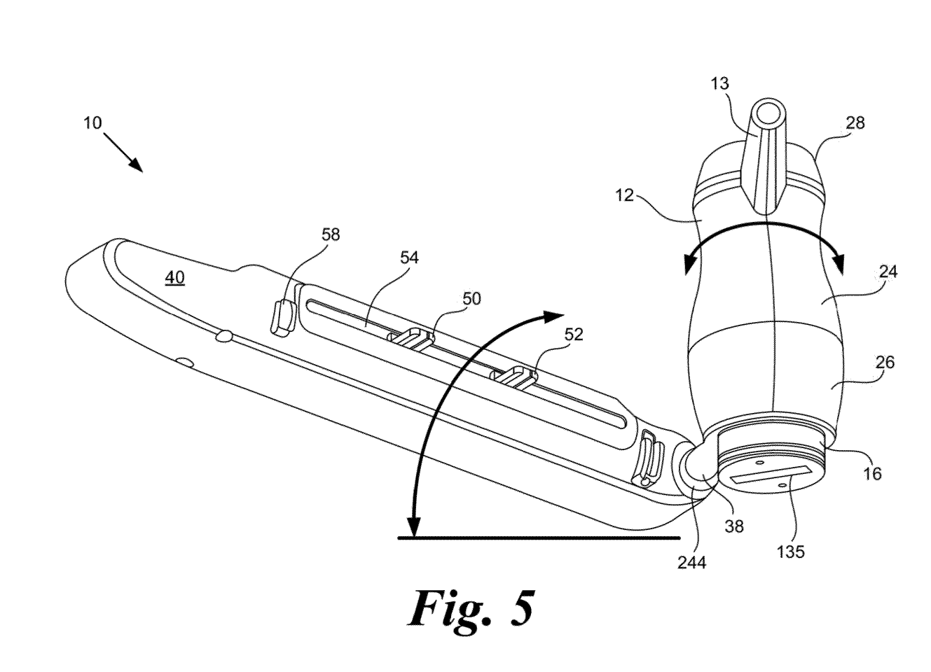

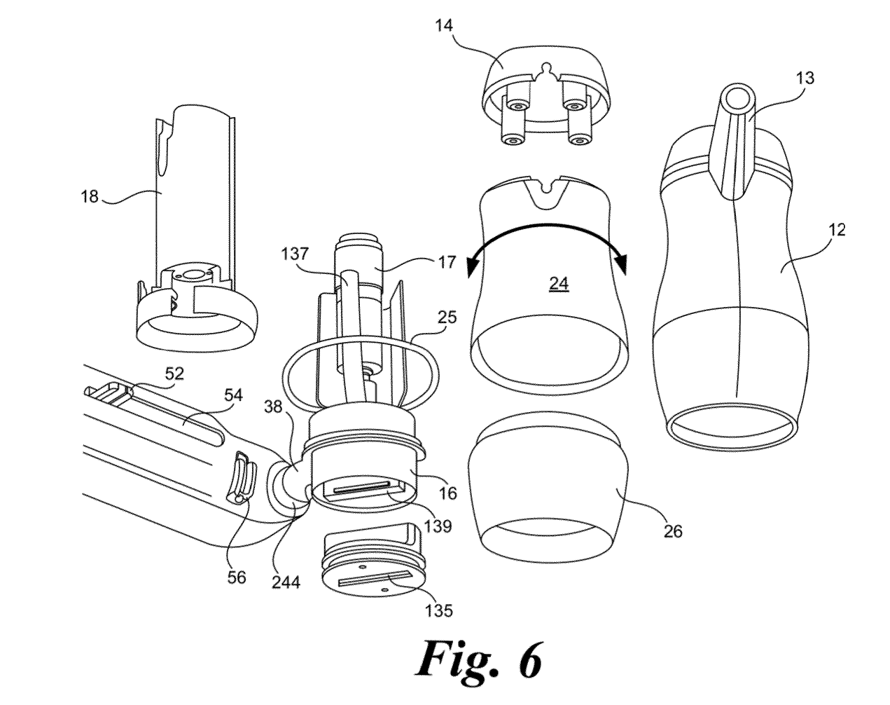

FIG. 1 schematically depicts a blood vessel access handset 10 that images blood vessels utilizing B-mode based single scan planes and/or rotationally-configured scan plane arrays. The blood access device includes an ultrasound transceiver housing 12 in communication with a central processing unit (not shown here but more fully described in FIG. 4 below) via power and data communication cable 13. The transceiver housing 12 includes a swiveling portion described in FIGS. 5 and 6 below. The swiveling portion swivels to accommodate the transceiver housing 12 to be grasped by righted-handed or left-handed users. Transceiver top 14 helps to secure the inner components within the transceiver housing 12 that is more fully described in FIG. 5. At the bottom is transducer support 16. Attached in pivotable contact with the transducer support 16 is a friction hinge housing 38 that connects injector arm 40 to the transceiver housing 12 via the transducer base 16. The injector arm 40 is equipped with a controller 47 having a rearward-located pushbutton control 42, a forward-located pushbutton control 44, and a 4-way toggle control 46. In signal communication with the push and toggle buttons 42, 44, and 46 of controller 47 are motorized moveable platforms 50 and 52 that slidably transit along the length of slot 54. Rearward control 42 retracts the moveable platform 50 away from the patient's targeted blood vessel independently of the position of the moveable platform 52. Forward control 44 moves the moveable platform 52 towards the patient's targeted blood vessel independently of the position of the moveable platform 50. With reference to FIG. 28 below, the 4-way toggle control 46 synchronously moves both the moveable platforms 50 and 52 synchronously together toward the patient's blood vessel if toggled towards the patient, and together away from the patient's blood vessel if toggled away from the patient. Adjacent to the slot 54 are cassette holders 56 and 58. As shown here the motorized platforms 50 and 52 occupy the distal third portion of the slot 54 away from the patient and are denoted as the “home” or start “position” within slot 54.

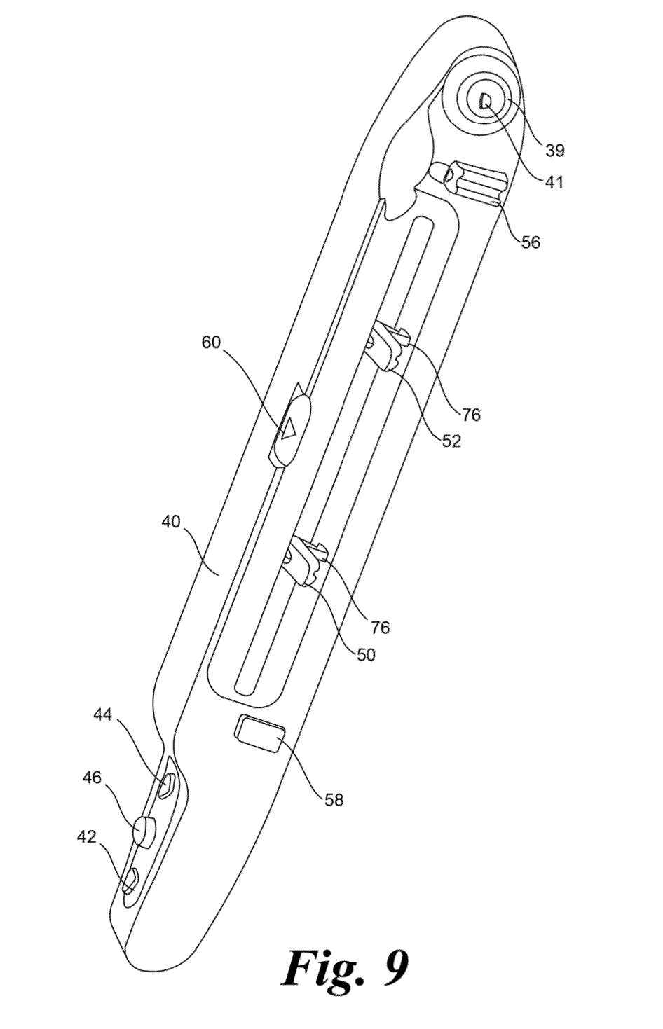

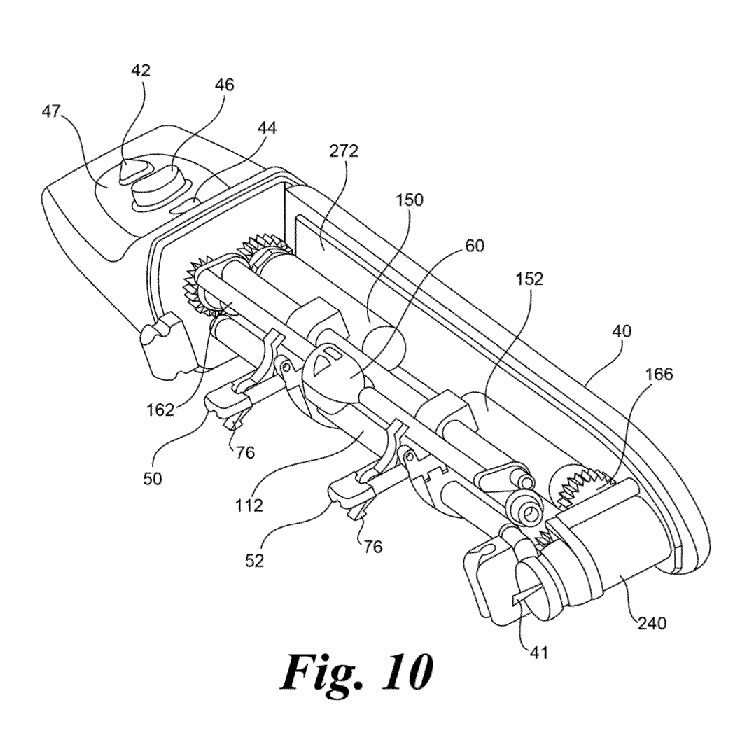

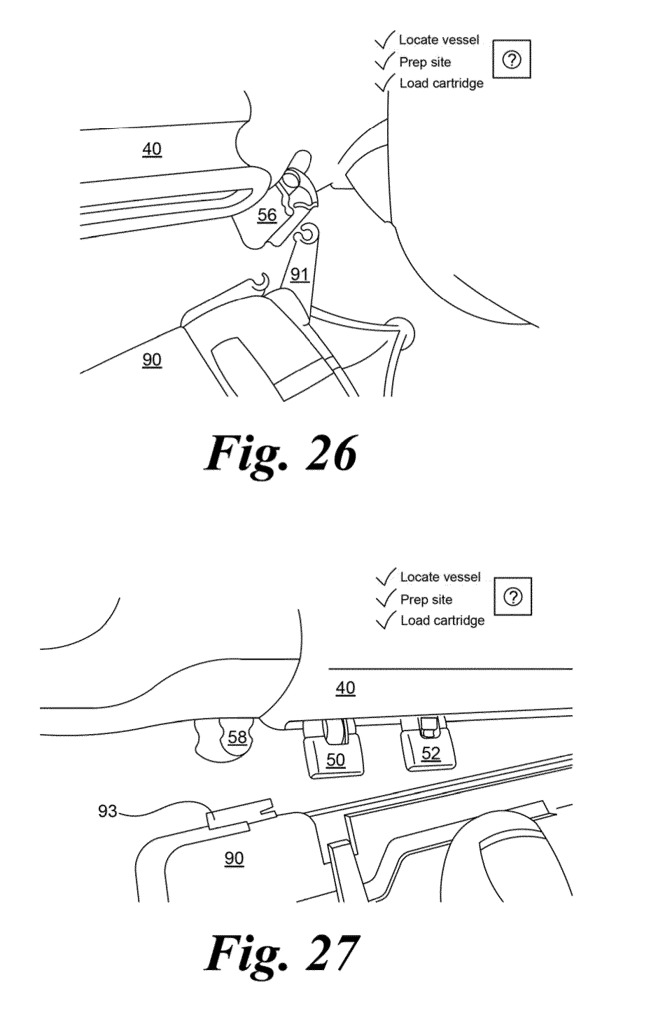

FIG. 2 schematically depicts the handset device 10 of FIG. 1 equipped with a detachably attachable cartridge or cassette 90 to the slot 54 side of the needle injector arm 40 by engagement with cassette holders 56 and 58 and moveable platforms 50/52 as described more fully in FIGS. 26 and 27 below. Referencing FIGS. 41 and 42 below, moveable platform 50 detachable engages with slideable needle mount 92 and moveable platform 52 detachable engages with slideable cannula mount 98 when the slideable mounts 92 and 98 are positioned within the cassette 90 in the “home” or “start” locus that is dimensionally accommodating or orientationally equivalent to the “home” and “start” positions of the motorized platforms 50 and 52 described in FIG. 1 above. As depicted in FIG. 2, cartridge 90 includes needle guide 94 at the end near the support base 16. The needle guide 94 forms an aperture from the combining of two half-apertures, one each from each swing door 105, such that when the two swinging doors 105 are in the closed position (shown in FIG. 41 below), each swinging door 105 has half of the aperture 94 (shown in FIG. 42 below) so that when the swinging doors 105 closes, the two aperture halves combine to form a single whole aperture to serve as the needle guide 94. The aperture of the needle guide 94 serves to prevent significant sideways slippage of the needle 120 and/or cannula 140 (discussed below) while proceeding through the needle guide's 94 aperture. Referencing again FIGS. 41 and 42 below, emerging from the needle guide 94 will be the needle 120 with overlapping cannula 140. As shown in FIG. 2, the mounted needle 120 is depicted as a pair of dashed lines suspended internally within the cassette 90. The cutting or piercing beveled end of the needle 120 is shown to occupy the portion of the internal space defined by the cassette's 90 swing door 105 when the slideable needle/cannula mounts 92/98 are in their home or start positions. Used cassettes 90 may be easily detached from injector arm 40 by pressing cartridge release button 60 that upon pressing by the user causes the moveable platforms 50 and 52 to pivot open release clips 76 (shown in FIG. 9 below) and thus disengage from the cartridge 90. The mechanism for cassette's 90 releasing action is more fully described in FIGS. 9 and 10 below.

FIG. 3 schematically depicts the handset 10 placed on a patient's arm. The handset 10 includes the cassette 90 attached to the slot 54 side of the injector arm 40 during a cannula placement operation into the patient's peripheral vasculature. In this illustration the transceiver housing 12 is pivoted for right-handed holding of the transducer support 16 against the patient's arm. The left hand of the user operates the tilting of the injector arm 40 about the friction hinge housing 38 and operation of the push and toggle buttons 42, 44, and 46 of controller 47 depicted in FIG. 1 above.

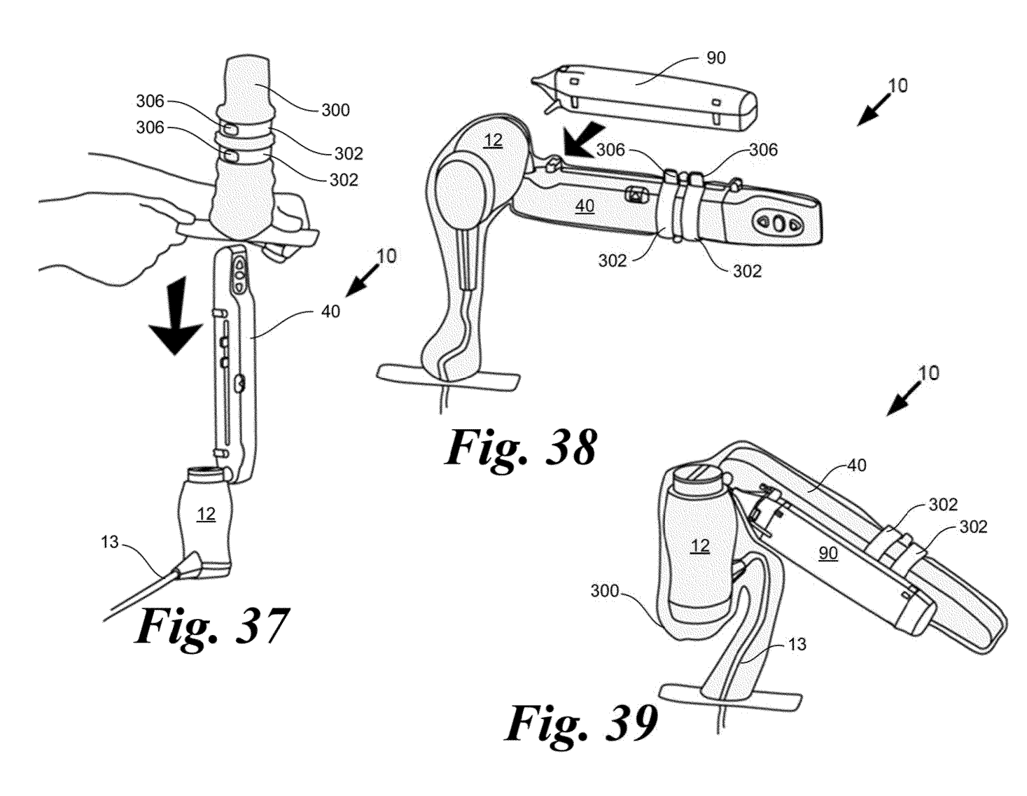

FIG. 4 schematically depicts an embodiment of a blood vessel access system deployed from a movable cart 200. The cart 200 includes a monitor 206 equipped with a touch sensitive screen 208, the monitor 200 being supported by an articulated arm 210 extending from a countertop 215 from which the access device 10 can be prepared for various blood access procedures undertaken within clean, aseptic, or sterile arenas. The power supply and communication cable 13 can conveniently access a computer having a central processing unit 202 operating within cart support 204. The central processing unit is configured to receive and process echoes of ultrasound signals to present images of insonified vasculatures. Alternatively the central processing unit may be built into the monitor 206. Included in the countertop 215 is a handset holder 217 shaped to hold the transceiver housing 12 bottom side up so that the transceiver housing's 12 support base 16 faces upward to conveniently allow application of a sterile transducer cap 270 shown in FIGS. 34-36 below or application of a sterile sheath 300 to envelop the handset device 10 illustrated in FIGS. 37-39 below. The cart 200 with access handset device 10, monitor 206, and central processing unit 202 may be conveniently rolled via wheeled extensions 220 nearby the patient to conduct blood vessel access procedures under clean, aseptic, or sterile arenas.

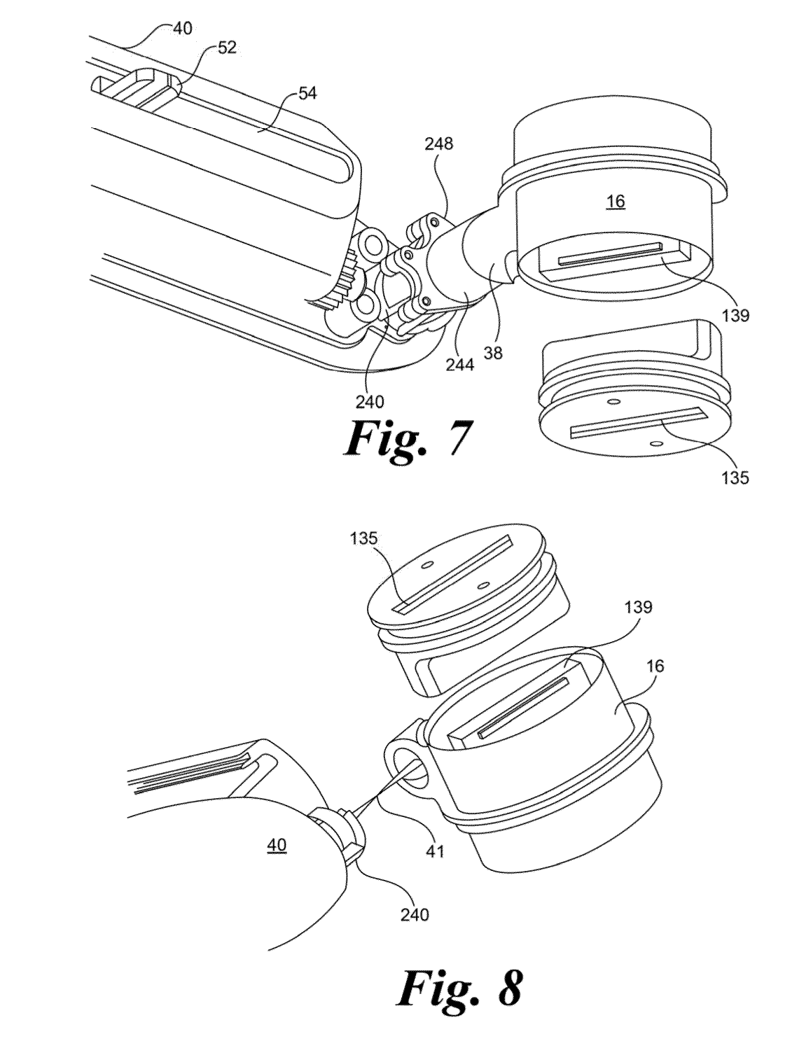

FIG. 5 schematically depicts a bottom perspective view of the handset 10 depicted in FIGS. 1-3. Ultrasound transceiver housing 12 may be swiveled for left or right handed holding as depicted by an arrow in FIG. 5 via swivel grasp 24 that slidably rotates about transceiver base 26. As depicted by an arrow in FIG. 5, the friction hinge housing 38 serves to anchor the pivotable injector arm 40 at a user-selected inclination relative to the rotational transducer 135 or to the patient's blood vessel under consideration for cannulation. In this depiction the motorized platforms 50 and 52 have changed positions with slot 54 from the home or start position depicted in FIG. 1 in that platforms 50 and 52 have slid more forward towards the transceiver housing 12. Attached with the hinge housing 38 is an exterior portion of the hinge shaft 244 that provides a conduit for a portion of the friction hinge 41 to occupy that is more fully described in FIGS. 7 and 8 below.

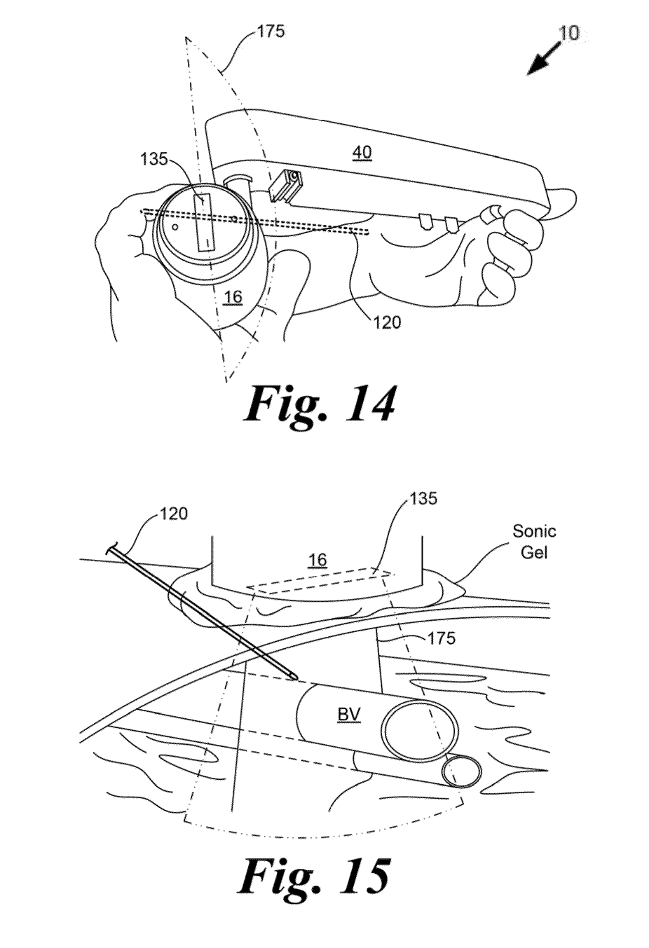

FIG. 6 schematically depicts components within transceiver housing 12. As depicted in FIG. 6, the transceiver housing 12 includes a swivel grasp 24 located between transceiver base 26 and transceiver cap 14. The power and communication cable 13 routes through the swivel grasp 24 and rotates to permit left-handed or right-handed holding during blood vessel and cannulation procedures. Between the swivel grasp 24 and transceiver base 26 is gasket 25. Located within the swivel grasp 24 is motor mount 18 from which transducer rotator motor 17 resides to pivotably rotate ultrasound transducer 135 upon user engagement of 4-way toggle switch 46 depicted in FIG. 1 above and described with regards to FIGS. 14, 16, 28 and 29 below. Extending from the motor 17 is electrical cable 137 that provides signal and power connection to the transducer 135 via connector block 139.

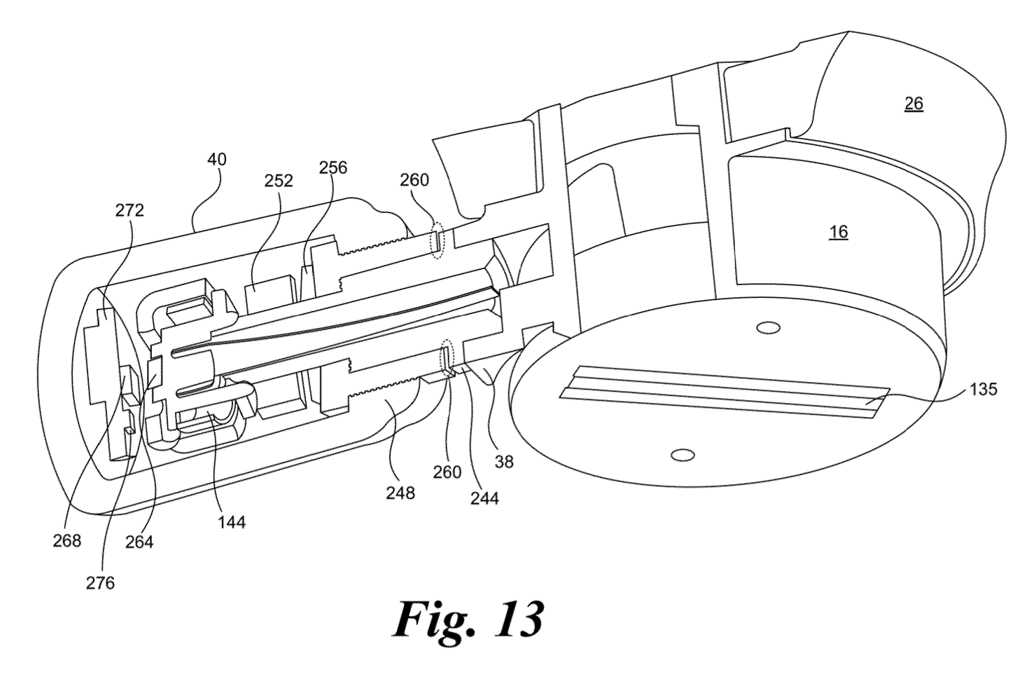

FIG. 7 schematically depicts a cutaway perspective view of the friction hinge housing 38 spanning and coupled to injector arm 40 and transducer base 16 depicted in FIGS. 1, 5, and 6 above. The hinge housing 38 extends from the transducer base 16 and is connected with the shaft portion of the friction hinge 41 more extensively shown and described in FIGS. 12 and 13 below. A hinge arm rotator 244 having three toed footing is secured to the arm 40. The hinge arm rotator 244 pivotally rotates with injector arm 40 about stationary hinge shaft 244. A hinge shroud 240 houses the friction hinge 41 more fully described in FIG. 12 below. The internal region of the hinge shroud 240 secures one end of the friction hinge 41 so that twisting forces caused by pivoting the injector arm 40 are conveyed to the hinge 41.

FIG. 8 schematically depicts another cutaway view of the friction hinge housing 38, and illustrating friction hinge 41 connecting injector arm 40 and transducer base 16. In this depiction portions of the hinge shroud 240 are removed to reveal sections of the friction hinge 41. One end of the friction hinge 41 is secured to the injector arm 40 via the shroud 240 and the other end to a slot located within the transducer base 16. Twisting forces are then conveyed into the friction hinge 41 by rotation of the injector arm 40 relative to the fixed transducer base 16.

FIG. 9 schematically depicts a perspective view of injector arm 40 on the slot 54 side. Upon pressing release button 60, release clips 76 open to disengage the moveable platforms 50/52 from cassette or cartridge 90 depicted in FIG. 1 above to reverse the attachment procedure depicted in FIGS. 26 and 27 below. Thus used cartridges 90 after a cannulation procedure may be easily detached from the injector arm 40 by pressing cartridge release button 60 that upon pressing by the user causes the moveable platforms 50 and 52 to pivot open release clips 76 and thus disengage from the cartridge 90.

FIG. 10 schematically depicts a cutaway perspective view of the injector arm 40 from the friction hinge region towards the controller 47 region. Mounts 50 and 52 slide along rail 112 respectively by electric motors 150 and 152 via their respective connection through gears 166. Controller board 272 receives signals from the controller 47 through its respective push and toggle buttons 42, 44, and 46 and delivers them for either independent operation of the electric motors 150 and 152 or synchronous operation, of motors 150 and 152 more fully described in FIGS. 28 and 29 below. Circuit board 272 also receives signals from push 42, 44 and toggle 46 buttons of controller 47 and delivers signals to transducer rotation motor 16 to cause rotation of the rotatable transducer 135. Pressing release button 60 engages press bar 162 that opens release clips 76 to cause disengagement of moveable platforms 50/52 from their respective engagement with cassette's 90 slideable mounts 92 and 98.

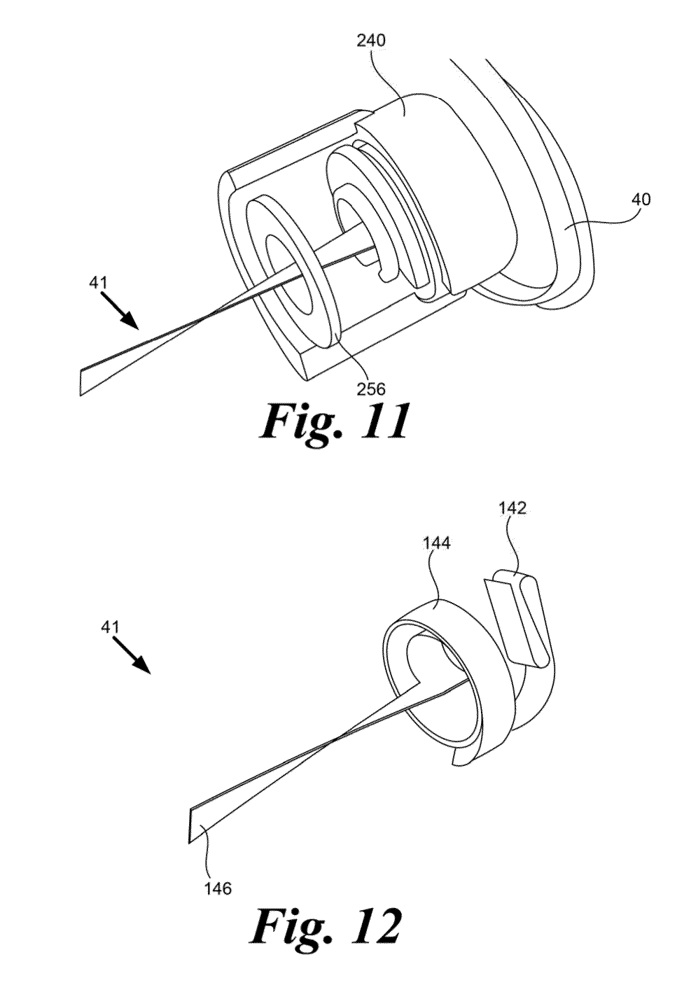

FIG. 11 schematically depicts a close-up cutaway view of the component parts within friction hinge shroud 240. The friction hinge 240 is connected with injector arm 40 and rotates when the arm 40 rotates. Torsional resistance to rotation is conferred by the terminal end of the hinge 41 described in more detail in FIG. 12 below. The middle portion of friction hinge 41 extends through spacing washer 256.

FIG. 12 schematically depicts friction hinge 41 removed from its shroud 240 shaped holder. The friction hinge 41 includes a shroud anchor 142, a coiled region 144, and a stator region 146. The shroud anchor 142 is mounted to the injector arm 40. Upon rotation of the arm 40, the shroud 240 rotates, and via the attached shroud anchor 142, causes a twisting action onto the coiled region 144 when the coiled region 144 undergoes tightening, or similarly, an uncoiling action when the arm 40 rotates in a reverse direction to uncoil or loosen the coiled region 144. The terminal end of the stator region 146 is mounted to a transducer base extension 16 shown in FIG. 13 below. The stator region 146 connected with the transducer base extension 116 within the transceiver housing 12 does not freely rotate with the pivotable action applied by the user to the injector arm 40 and by mechanical extension to the friction hinge 41 holder or shroud 240. The end of the stator region 146, by being held by transducer base extension 116 provides a clamping resistance to twisting motion forces conveyed by the coiled region 144. The coiled region 144 lessens strain conveying forces received by the stator region 146 to minimize fatigue or avoid fracturing of the stator region's 146 connection to the transducer base extension 116.

FIG. 13 schematically depicts a cross-sectional perspective view of the injector arm 40 in the region of the friction hinge housing 38 that provides pivotable connection between the injector arm 40 and the transducer base 16 of the transceiver housing 12. The friction hinge 38 pivotably connects the injector arm 40 to the transceiver housing 12 and allows the user to change the angle at which the needle enters the patient's tissue, convey positional information to the central processing unit 202, and keep in position the injector arm 40 at a user-selected angle while moving the needle 120 with overlapping cannula 140 towards the patient, within the patient, or away from the patient. The friction hinge housing 38 is connected to the non-rotatable hinge shaft 244. A threaded hinge arm 248 secures the hinge shaft 244 by variable tightening caused by turning the threaded hinge arm 248. Together with a securing nut 252 and spacing washer 256, the amount of turning resistance and self-holding ability is conferred to the injector arm 40 allowing it to stay self-standing or supporting at a user-selected angle relative to the patient. The hinge arm nut 252 rotates as the injector arm 40 rotates, and with this rotation, the hinge shroud 240. The contacting surfaces depicted in the dashed oval area 260 between the hinge arm rotator 248 and the hinge shaft 244 are configured to be friction-generating surfaces, so that combined with the adjustable tightening conveyed by the threading action of the threaded hinge arm nut 248 and securing nut 252, the forces necessary to restrain further pivoting of the injector arm 40 once positioned at a user-selected angle is obtained.

Attached to the hinge shroud 240 is a magnet 264. Changes in the magnets displacement caused by the rotation or pivoting of the hinge shroud 240 is detected by magnet sensor 268 attached to arm controller board 272. The changes in magnetic strength detected by magnet sensor 268 as a consequence of changing the position of the magnet 264 relative to sensor 268 changes the electronic signals produced by the sensor 268. The changes in magnetic induced signals permit determination of the rotation angle of the arm 40 relative to the transducer base 16 and/or transceiver housing 12. Magnetic induction signals conveyed to processor 276 configured with executable instructions having either a look-up table or microprocessor-readable instructions to execute linear and/or polynomial regression analysis to allow determination of the angle that the injector arm 40 presents relative to the transducer housing 16, the transceiver housing 12, and/or the ultrasound transducer 135. Alternatively, angle information of the injector arm 40 relative to the transducer support base 16, transducer 135, or transceiver housing 12 can be determined using computer executable instructions applied to the digitized versions of the magnetic signals conveyed from the magnet sensor 268, either to the microprocessor-equipped computer 202 conveyed through the signal lines located within power and data cable 13, or by local processing of the magnetic signals via the processor 276 located on the arm controller board 272.

FIG. 14 schematically depicts a perspective view of the bottom region of the handset 10 illustrating the transducer 135 emanating a scan plane 175 substantially perpendicular to the needle 120 that crosses through it. Here the rotatable transducer 135 of the bottom up illustrated transducer base 16 outputs a scan plane 175 substantially perpendicular to the needle 120. The cartridge 90 is not shown. The needle 120 is shown in dashed lines suspended in space.

FIG. 15 schematically depicts a perspective view of the scan plane 175 intersecting the across the short axis of blood vessel BV that shows the needle 120 poised to enter the anterior wall near the midline of the blood vessel BV. The needle 120 in relation to the ultrasound transducer 135 emanating a scan plane 175 that intersects it is substantially perpendicular to the short axis of blood vessels BV. Tissues exposed to the ultrasound energy scan plane 175 denotes an insonified region of the patient's vasculature from which continuous images of substantially a short axis cross-sectional view is presented on the monitor 206 in image depictions illustrated in FIGS. 20, 23, and 24. Sonic gel is used to acoustically couple the transducer 135 with the surface of the patient to more readily and efficiently convey ultrasound energy from the transducer 135 into the patient's vasculature and receive fundamental and harmonic ultrasound echoes returning from the patient's vasculature.

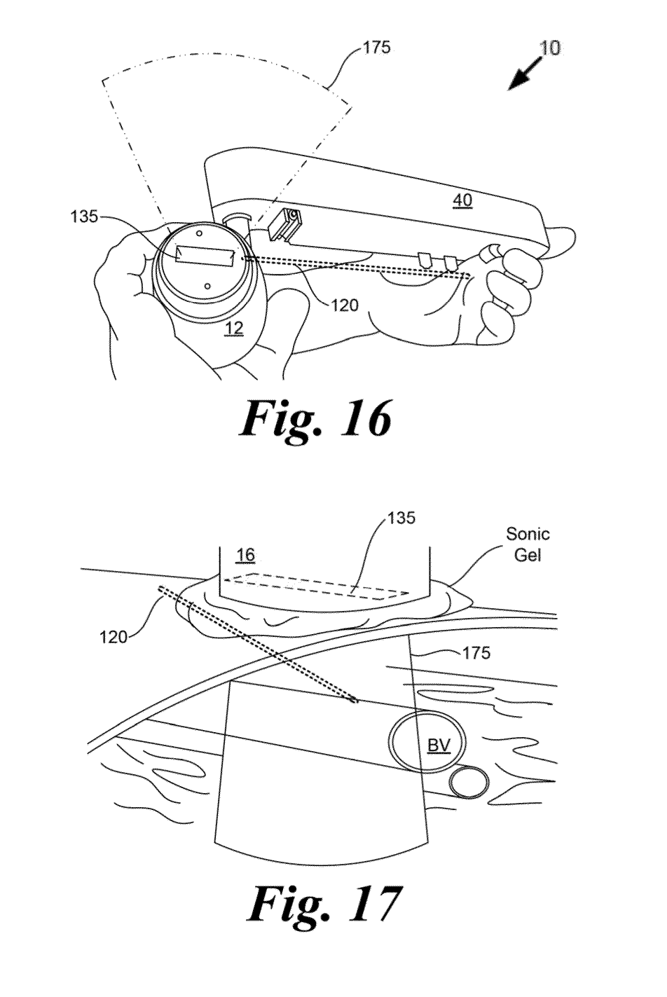

FIG. 16 schematically depicts a perspective view of the bottom region of the handset 10 illustrating the transducer 135 emanating a scan plane 175 substantially parallel to the needle 120 that transits within it. The cartridge 90 is not shown. The needle 120 is shown in dashed lines suspended in space. The substantially parallel transiting of the scan plane 175 by the needle 120 represents the long-axis cross-sectional configuration for an insonified region of the patient's vasculature when presented in long-axis view per FIGS. 25 and 30-32 depicted below.

FIG. 17 schematically depicts a perspective view of the scan plane 175 intersecting the across the long axis of blood vessel BV that shows the needle 120 poised to enter the anterior wall near the midline of the blood vessel BV. FIG. 17 schematically depicts a perspective view of the parallel configuration of the needle 120 in relation to the ultrasound transducer 135 emanating scan plane 175 that intersects substantially parallel to the long axis of blood vessel BV. Tissues exposed to the ultrasound energy scan plane 175 denotes an insonified region of the patient's vasculature from which images having a substantially long axis cross-sectional view is presented on the monitor 206 in image depictions illustrated in FIGS. 25, 30, 31 and 32. Sonic gel acoustically couples the rotatable transducer with the surface of the patient.

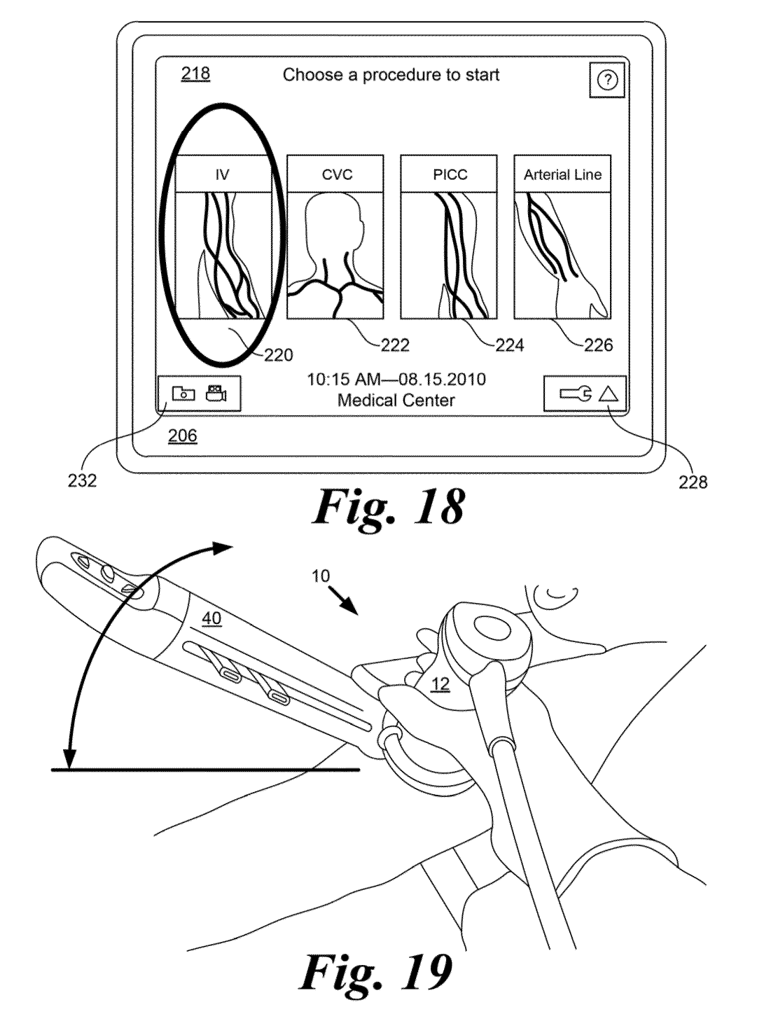

FIG. 18 schematically depicts the touch screen monitor 206 presenting a home screen 218 illustrating a panel of four blood vessel based access procedures characterized by different icons and acronyms. As stated previously, monitor 206 may be a touch screen. The panel of blood vessel access procedures includes a peripheral intravenous IV procedure 220, a central venous cava CVC procedure 222, a peripherally inserted central catheter PICC procedure 224, and an arterial line procedure 226. In the case of a touch screen monitor 206, the IV procedure 220 icon is touched by the user, indicated by the oval, to bring up menu items to conduct this blood vessel access procedure. Also shown are touch sensitive tool icon 228 and data output icons 232.

FIG. 19 schematically depicts the handset 10 surveying for a peripheral vein undertaken during the IV procedure selected from the home screen depicted in FIG. 18. The injector arm 40 can pivot freely from shallow acute angles to steep acute angles in relation to the transceiver 12 as denoted by the arrow in FIG. 19.

FIG. 20 schematically depicts an ultrasound image presented in screenshot 260 on the monitor 206 while surveying for a blood vessel undertaken during short axis mode when the scan plane 175 emanating from the transducer 135 depicted in FIG. 15 intersects blood vessels substantially at a perpendicular orientation. Screenshot 260 includes a contrast icon 262, a still capture icon 264, a movie capture icon 266, a home return icon 268, and a return to prior screen icon 269. In this screenshot example of an insonified vasculature image, a center located blood vessel is presented in short axis cross section when the position of the rotatable transducer is indicated to be in short axis mode by the presence of a short axis icon 282, depicted in as a thick circle. Appearing above the short axis blood vessel BV, another blood vessel BV is depicted substantially in a long axis cross sectional view. Applied to the ultrasound image of screenshot 206 is an overlay having positional information in the form of a vertical axis line 281 and a horizontal axis line 286 located at 20 degrees that can be varied in its position depending on the tilting angle that the user adjusts the injector arm 40 to occupy. In this screenshot the vertical axis line 281 is shown bisecting the center-located short axis-presented blood vessel BV and represents the approximate location of the rotatable transducer 135 of handset 10. Perpendicular to and intersecting with the vertical axis line 281 are three horizontal lines 286, 290, 294 indicating various inclination angles of the injector arm 40 to achieve different penetration depths for needle injection and cannulation. Horizontal axis line 286 represents a depth when the injector arm presents, for example, a 20 degree inclination angle and horizontal axis line 290 defines when the injector arm presents, for example, a 60 degree inclination angle. Between these two lines 286 and 290 is horizontal axis line 294 that represents a depth or is indicative when the injector arm occupies a 33 degree inclination angle relative to the transducer 135. The intersection of any given horizontal axis line, seen in this example as horizontal axis lines 286, 290, or 294 with the vertical axis line 281 represents the cross-hair like locus or sighting aid position where cutting bevel end 123 (shown in upper inset of FIG. 28A below) of the needle 120 is expected to appear as the needle 120 advances while the arm 40 is at, for example, a 20 degree penetration angle, a 60 degree angle, and a 33 degree angle. Thus any vertical and horizontal axis intersection serves as cross-hair like sighting aid for the positional overlay when the screenshot image is presented in short axis cross-sectional views. The horizontal axis 286 can be adjusted to intersect at any given location of the vertical axis 281 indicative of the location of the transducer 135 by tilting or pivoting the injector arm 40 while holding the transceiver housing 12 firmly against the patient's skin. In this example, the intersection of horizontal line 294 with vertical line 281 is near the midline portion of the anterior wall of the short-axis cross-sectional view of blood vessel BV. Generally, penetration of the blood vessel by the needle 120 near the midline of the anterior wall represents a good position to initiate needle injection and cannulation procedures.

Commonly the angle of inclination of the injector arm 40 is set for penetration such that the vertical and horizontal crosshairs would be intersecting at the anterior wall along the midline of the targeted blood vessel when the image and image overlay is presented in short-axis cross-sectional views. The anterior wall of the blood vessel is the wall that is closer to the rotatable transducer 135. Also presented in screenshot 260 is vessel access menu 280. Access menu 280 may be configured for drop down presentation and includes the steps of 1, locating the target vessel (Locate Vessel); 2, prepare the site (Prep Site); 3, load cartridge 90 onto injector arm 40 (Prep Cartridge); 4, cannulate the target vessel (Cannulate), and 5, document the procedure (Document).

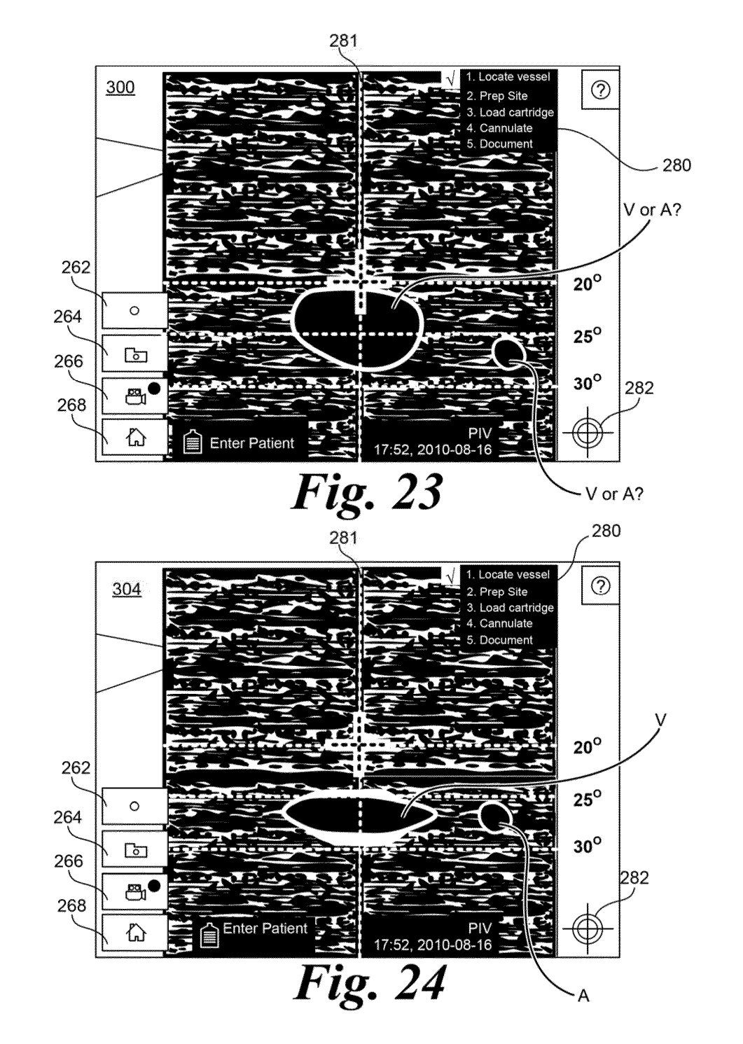

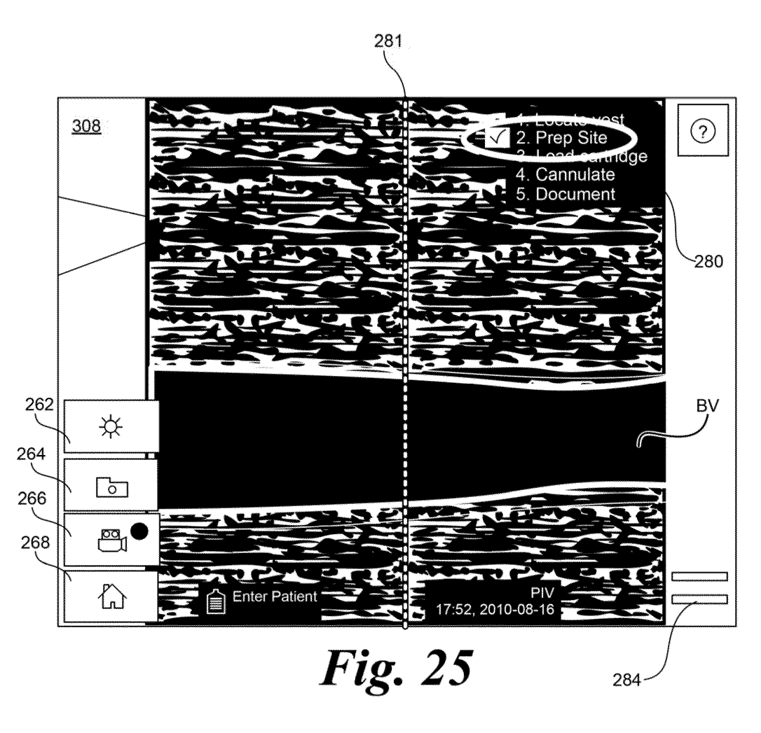

FIGS. 21 and 23 schematically depict the differential collapsibility or compressibility of veins and arteries when subjected to pressure of the transceiver 12 pressing on the patient's arm. Generally, thinner walled veins will collapse while thicker walled arteries will retain their substantially circular cross-sectional shape. The user may use this characteristic of veins and arteries to identify the target vessel.

FIGS. 23 and 24 schematically depict the differential compressibility of veins and arteries as presented on screen images on touch screen 208 under the “Locate vessel” procedure of the access menu 280. FIG. 23 illustrates in screen shot 300 two blood vessel in cross-section, one large having its anterior wall located near 20 degrees and the other small having its anterior wall located near 25 degrees. Both blood vessels are substantially circular and may be a vein V or an artery A. The unknown nature of these small and large blood vessels are designated as “V or A”, that is, “vein or artery”. FIG. 24 schematically depicts in screenshot 304 the results of ascertaining the blood vessel type upon application of a downward force from the ultrasound transceiver 12. The larger of the two blood vessels collapses substantially from the location at the 20 degree cross hair position when receiving the downward force and the smaller blood vessel remains substantially un-collapsed or is not distorted substantially as the anterior wall remains close to the 25 degree location in the non-compressed state. The larger blood vessel is designated to be a vein V and the smaller blood vessel to be an artery A since the vein V collapses more so than the artery A upon exposure to the downward force. In other embodiments of the handset 10, the rotatable transducer 135 may be configured for Doppler based ultrasound utilizing sound analysis of the patient's pulsation and blood flow to confirm the venous or arterial nature of the targeted blood vessel.

FIG. 25 schematically depicts an ultrasound image presented in screenshot 308 of a long axis view of a targeted blood vessel BV presented on the monitor 206. The long axis view is indicated by icon 284 which shows the walls similar to a tube presented in long axis. The axis menu 280 is check marked to “Prep Site”. For site preparation that will depend on whether the needle injection and subsequent cannulation is to be undertaken within clean, aseptic, or sterile arenas. Once the blood vessel is found, be it artery or vein, generally the cartridge 90 is fitted to the injector arm 40 and the user proceeds to the next step indicated as “cartridge”.

FIGS. 26 and 27 schematically depict the attachment or loading of the cannula cartridge 90 to the slot 54 side of injector arm 40. In FIG. 26, attachment post 91 extending from the cartridge 90 is removeably attachable with the cartridge holder 56 and provides for pivotal alignment with the slots (not shown) located within slideable platforms 92/98 (not shown) and the cartridge's 90 clip 93 with cartridge holder 58. Thereafter, upon swinging into alignment from pivotable attachment to cartridge holder 58, the slots of the slideable platforms 92/98 (not shown) are engaged with the moveable platforms 50/52 followed by the rear portion of the cartridge 90's attachment clip with the arm 40's holder 56.

FIGS. 28A-29B schematically illustrate the “cannulate” step represented in access menu 280 and sets forth how the controller's 47 push and toggle buttons 42, 44, and 46 are used in a needle injection and cannulation procedures employing the cassette 90 that is mounted to the slot 54 side of the injector arm 40. The injector arm's 40 moveable platforms 50 and 52 are respectively removeably connected with the slideable mounts 92 and 98 that respectively hold the needle 120 and cannula 140. The moveable platforms 50 and 52 respectively drive the slideable mounts 92 and 98. The “cannulate” procedure involves needle 120 injection and cannulation of a user-selected blood vessel with the cannula 140 that is in slideable connection with the needle 120. The “cannulate” step is the procedure that may be chosen after loading the cartridge 90 as shown in FIGS. 26 and 27 above. As shown in the access procedure menu 280 presented on the monitor 206 visible to the handset 10 operating user, the “cannulate” step occurs after the “load cartridge” step. Also illustrated in FIG. 28A is that substantially perpendicular toggling of the 4-way toggle button 46, that is tilting approximately 90 degrees upwards or downwards from the long axis of the injector arm 40, results in the single rotation movements of the rotatable transducer 135 as shown in FIGS. 14 and 16 above to easily permit the user to switch between short-axis and long-axis cross-sectional views of the insonified based images being presented on the monitor 206.

In more detail FIG. 28A schematically depicts an example of the independent and synchronous movement of the slideable mounts 92/98 within the cartridge 90 with reference to cartridge's 90 swinging doors 105 that remain fixed in place. The independent and synchronous movement of the slideable mounts 92/98 is driven by the motions of the moveable platforms 50/52 that in turn respond to the handset 10 user engagement of the controller's 47 push and toggle buttons 42, 44, and 46 in response to the user's viewing of monitor presented images. There are three scenarios depicted for the slideable mounts 92/98 with reference to the closed swinging doors that are co-aligned vertically to represent the cartridge 90 being fixed-in-place to the slot 54 side of the injector arm 40 previously illustrated. The first scenario shown in the top depiction represents the slideable mounts occupying a “home” or “start” position in which the slideable mounts are adjacently touching and the bevel 121 and cutting tip 123 of the needle 120 resides just inside orifice 94. That is, the cutting tip 123 does not protrude from the cartridge's 90 orifice 94. In this “home” position the adjacently touching slideable mounts 92/98 have no space between them resulting in this illustration with the end of the cannula 140 just behind the rearward end of the bevel 121, as shown in the inset.

The second scenario, involves the needle 120 with overlapping cannula 140 protruding deeply beyond the orifice 94 formed by closed swinging doors 105 for injection into a deeply located blood vessel. As shown in the upper middle depiction the user tilts toggle button 46 in the direction towards the patient or orifice 94 indicated by radial lines around toggle button 46 and the smaller direction arrow aimed towards the orifice 94. The forwardly toggling direction or tilting of toggle 46 towards the orifice 94 is substantially parallel to the long axis of injector arm 40. Here both slideable mounts 92/98 advance equally forward synchronously towards the orifice 94 to protrude the bevel region 121 of needle 120 having the same cannula 140-to-needle bevel 121 relationship as shown in the first or “home” scenario above.

The third scenario, involves both the needle 120 with overlapping cannula 140 both retracted the same distance from the more protruding second scenario discussed above. As shown in the lower middle depiction the user tilts toggle button 46 in the direction away from the patient or the orifice 94 indicated by radial lines around toggle button 46 and the smaller direction arrow aimed away from the orifice 94. The rearward toggling direction or tilting away of the toggle 46 from the orifice 94 is substantially parallel to the long axis of injector arm 40. Here both slideable mounts 92/98 advance equally rearward synchronously away from the orifice 94 to protrude the bevel region 121 of needle 120 less deep than the second scenario above. As with the second scenario, the third scenario maintains the same cannula 140-to-needle bevel 121 relationship as shown in the first or “home” scenario above.

Still referencing FIG. 28A, the fourth scenario involves retracting the needle's 120 bevel 121 into the cannula 140 by engagement of rearward control 42 to cause the rearward displacement of slideable needle mount 92 away from the orifice 94. The forth scenario is shown in the bottom depiction wherein the user presses rearward control button 42, indicated by radial lines around button 42, to cause the rearward motion of slideable mount 92, that is, movement away from the patient or away from the orifice 94 of closed swinging doors 105. This rearward motion of slideable mount 92 caused by the user's engagement or pressing of rearward button 42 occurs independently from the existing position that the slideable mount 98 currently occupies. This rearward motion continues until the user stops pressing rearward button 42, and a space or gap G is created between slideable mounts 92 and 98, and in direct proportion to the rearward motion. The inset of the bottom depiction shows that the same space or gap G created between the slideable mounts 92 and 98 is the same gap G space that the now retracted needle 120 bevel 121 is withdrawn into the lumen of the cannula 140. That is, with the needle's 120 bevel 121 withdrawn deeper into the cannula 140, there is a different cannula 140-to-needle bevel 121 relationship as shown in the inset of the lower depiction or fourth scenario than the bevel 121-to-cannula 140 relationship of the first or “home” scenario depicted in the first scenario above.

In greater detail FIGS. 28A and 28B schematically depict the interplay of controller 47 operations with regards to the motorized forwardly directed co-movement of the needle assembly via slideable needle mount and cannula assembly via slideable cannula mount 98 undertaking during the cannulate step of access menu 280. Referencing FIGS. 1 and 2 above, the moveable platform 50 detachably engages with the cassette's 90 slideable needle mount 92 and the moveable platform 52 detachably engages with the cassette's 90 slideable cannula mount 98. The slideable needle/cannula mounts 92 and 98 have slot receptacles (not shown) that accommodate and hold the rectangular shapes of the moveable platforms 50 and 52. To the slideable needle mount 92 is mounted needle 120 and to the slideable cannula mount 98 is mounted cannula 140. As shown here the beveled or pointed or cutting surface of the needle 120 extends beyond the internal end of the cannula 140. Referencing FIG. 23 in view of FIGS. 2 above and 24 below, moveable platform 50 detachable engages with slideable needle mount 92 and moveable platform 52 detachably engages with slideable cannula mount 98 when the slideable mounts 92 and 98 are positioned within the cassette 90 in the “home” or “start” position that is dimensionally accommodating to the “home” and “start” positions of the motorized platforms 50 and 52 described in FIG. 1 above. Emerging from the needle guide 94 and into the tissue beneath the transducer base 16 and into the patient's tissue is the needle 120 with overlapping cannula 140. The cutting or piercing beveled end 123 of the needle 120 is shown approaching and about to pierce the anterior wall of the blood vessel BV (left side drawing) to enter the vessel's lumen by the forward motion of the slideable needle/cannula mounts 92/98 as engaged by forward motion tilting of controller 47 toggle button 46. With the injection or penetration of the needle 120 into the vessel's lumen, the overlapping cannula 140 enters with and just behind the cutting edge of the needle 120. Here the blood vessel is depicted in long axis cross-section (right sided drawing).

The inset in the left side drawing of FIG. 28B illustrates a particular embodiment in which the squared-off truncated end of the cannula 140 is immediately behind the rear portion of the beveled end 121 of the needle 120 so that cannulation of narrow lumen blood vessels can be undertaken. The placement of the blunt end of the cannula 140 immediately behind the rear portion of the beveled end 121 is controlled by the movement of the slideable needle mount 92 that is pushed against the slideable cannula mount 98 when engaged in synchronous forward movement by the forward pushing of the 4-way toggle control 46 to advance both the needle 120 and cannula 140 synchronously towards the blood vessel at the same displacement rate. In particular embodiments, the cartridge 90 comes pre-configured in the “home” position wherein the slideable needle/cannula mounts 92/98 are adjacent and nearly abutting to each other so that synchronous forward traveling motion toward the patient's blood vessel of the slideable needle/cannula mounts 92/98 keeps the squared-off truncated end of the cannula 140 immediately behind the rear portion of the beveled end 121 of the needle 120 so that the cannula 140 does not cover over the cutting surfaces 122 while entering the patient's skin or when approaching or attempting to pierce through the anterior wall of the patient's targeted blood vessel selected for cannula placement. The inset is similar to the co-advancing and equal speed advancing of the needle 120 with overlapped cannula 140 described in the first scenario or top depiction illustrated in FIG. 28A above.

Similarly in greater detail FIGS. 29A and 29B schematically depict the interplay of controller 47 operations with regards to implementing separate and independent motorized movement of the needle assembly or slideable needle mount 92 and the cannula assembly or slideable cannula mount 98. As previously depicted in FIG. 28A above, under the cannulate procedure of access menu 280, FIG. 29A depicts separate movement and independent movement of the needle 120 via slideable mount rearward motion by the user pressing rearward button 42. Separately, the forward movement towards the orifice 94 of the cannula 140 occurs via the user pressing forward button 44, thus protruding the cannula 140 substantially beyond the orifice 94. With the separate pressing of rearward button 42, the bevel 121 of needle 120 is withdrawn deeply inside the cannula 140 near the rearward portion of closed swinging doors 105. Forward motion of the slideable cannula mount 98 towards the orifice 94 is indicated by the right side downward angled motion arrow and rearward motion of the slideable needle mount 92 away from the orifice 94 is indicated by the left side upwardly angled motion arrow.

FIG. 29B schematically illustrates cannulation of a blood vessel BV presented in long axis mode via use of control buttons 42, 44, and 46. In the upper drawing is illustrated the synchronous movement of both cannula 140 and needle 120 by the user forwardly tilting the toggle button 46. In the middle drawing is illustrated the separate and independent motions of the needle 120 via the rearward button 42 and the cannula 140 with the forward button 44. The upper FIG. illustrates the overlapped cannula 140 with the needle's 120 bevel 121 was advanced and now penetrated through the anterior wall of the blood vessel BV to reside approximately just beneath the anterior wall of the blood vessel BV via the tilting of toggle control 46 towards the patient. As shown, the cutting tip 123 of the bevel 121 is sufficiently far from the posterior wall of the blood vessel. Thereafter, as shown in the middle drawing, the distal end of the overlapped cannula 140 is advanced beyond the cutting edge 123 of the bevel and now occupies a space close within the blood vessel close to the anterior wall. Also illustrated is the refraction of needle 120 from the blood vessel BV that is implemented independently of the forward advancing cannula 140 by the user independently pressing the rearward button 42. As shown in the middle drawing the bevel 121 occupies the space very close to and in the process of crossing the anterior wall Thereafter, touching pushbutton control 44 slides the cannula further off the needle 120 and deeper into the blood vessel BV lumen. The needle 120 may be refracted further away from the anterior wall of the blood vessel by pushing pushbutton control 42 away from the patient. In this way cannula 140 may be advanced within the lumen with a minimum of kinking

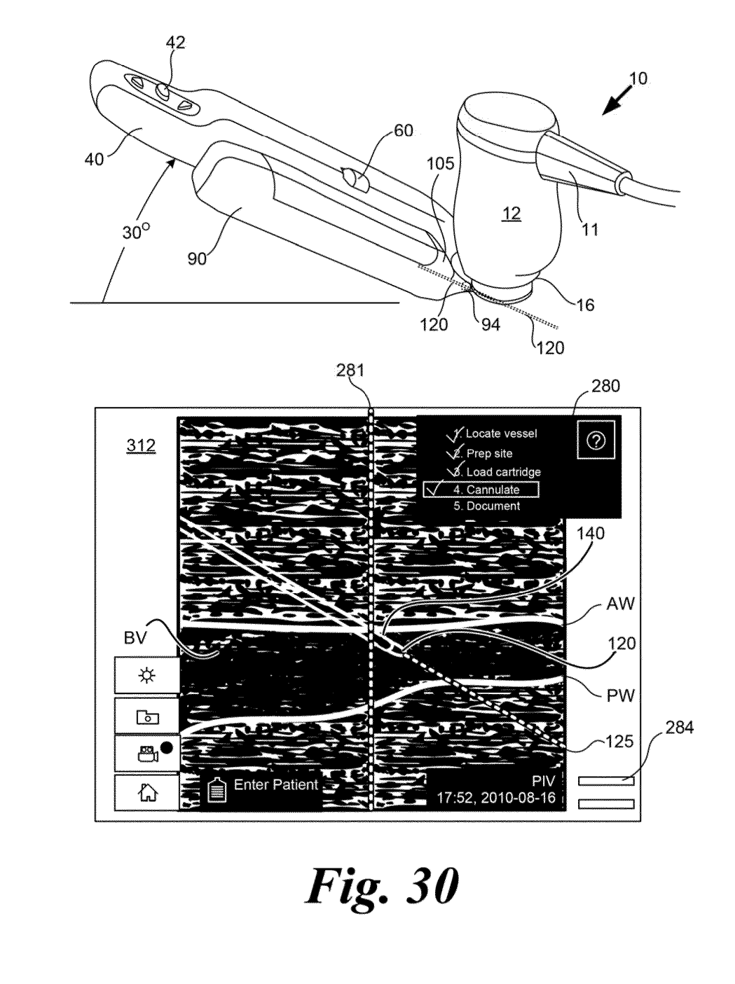

FIG. 30 schematically depicts a screenshot 312 in long-axis cross-sectional view when the penetration of the needle 120 with overlapping cannula 140 is seen to be penetrating through the blood vessel BV at 30 degrees relative to the rotating transducer 135 during the “cannulate” procedure of access menu 280. Applied to the ultrasound image of long-axis presented screenshot 312 is the overlay having positional information in the form of the vertical axis line 281 (also shown in short-axis presented screenshot 306 of FIG. 20 above) and a trajectory line 125 signifying the expecting pathway the needle 120 with overlapping cannula 140 will transit while the injector arm 40 remains at 30 degrees relative to the rotatable transducer 135. Here an image of the needle 120 with overlapping cannula 140 is shown penetrating the anterior wall AW of the blood vessel shown in long-axis mode as indicated by long axis icon 284. The needle tip 121 is kept near the lumen's midline when the injector arm 40 is, for example, approximately at a 30 degree angle relative to the base of the transducer 135. The cutting surface 123 (FIG. 29A) of the needle 120 is stopped or otherwise drawn back so as to not puncture the blood vessel's posterior wall PW. The angular change undertaken by the rotatable ultrasound transducer 135 between the short axis and long axis cross-sectional views may be less than ninety degrees, substantially ninety degrees, or greater than ninety degrees. The user may slightly rotate the transducer support 16 by pushing the injector arm 40 racially about the long axis of the transceiver housing 12 with one hand and holding the transceiver housing 12 firmly against the surface of the patient with the other hand, while maintaining the angular tilt or angular position of the injector arm 40 relative to the transducer 135 in order to reposition the rotatable ultrasound transducer 135 as needed to generate a sufficient long axis cross-sectional view of the blood vessel BV undergoing cannulation.

FIG. 31 schematically depicts a screenshot 316 in long-axis cross-sectional view when the blood vessel BV occupying needle 120 with overlapping cannula 140 adjusted for cannulation to 20 degrees relative to the rotating transducer 135 during the “cannulate” procedure of access menu 280. In screenshot 316 the positional information overlay contains a change of information relating to the re-adjustment of the injector arm 40 by the user just prior to commencing cannula 140 advancement and needle 120 retraction. In screenshot 316 the change in positional information of the positional information overlay is shown by the trajectory line 125 occupying a 20 degree tilt and displaced towards the right side of screenshot 316. The cutting point (123 shown in upper inset of FIG. 28A above) of the needle 120 is withdrawn to a point just inside the anterior wall AW.

FIG. 32 schematically depicts a screenshot 320 in long-axis view after cannulation of the blood vessel with cannula 140. Cannulation then proceeds by engaging toggle 46 to push the cannula beyond the needle's 120 bevel residing or spanning through the anterior wall/lumen interface. The last step, document, of procedure menu 280, involves recording the cannulation by the user touching still camera icon 264 or camera tool icon 266 and storing the still or video images on computer's 202 local hard drive, an attached flash drive, or alternatively on a network drive in communication with the computer 202. The needle may then be withdrawn from the patient's blood vessel BV and from the patient by engaging the rearward button 42, leaving the cannula 140 in place. As shown here the end of the cannula 140 is shown closer to the blood vessel's BV posterior wall PW than its anterior wall AW.

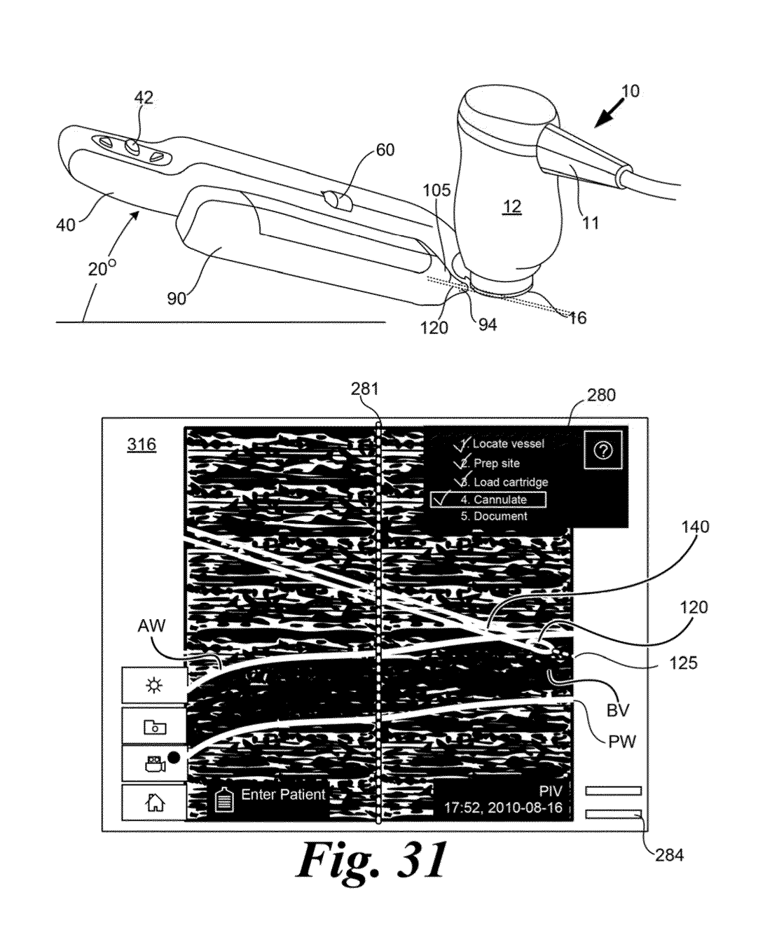

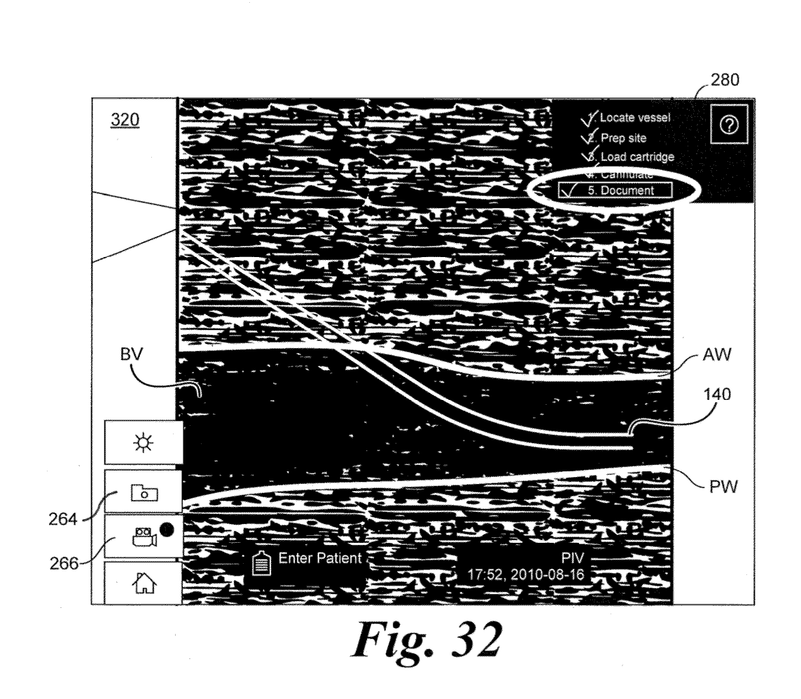

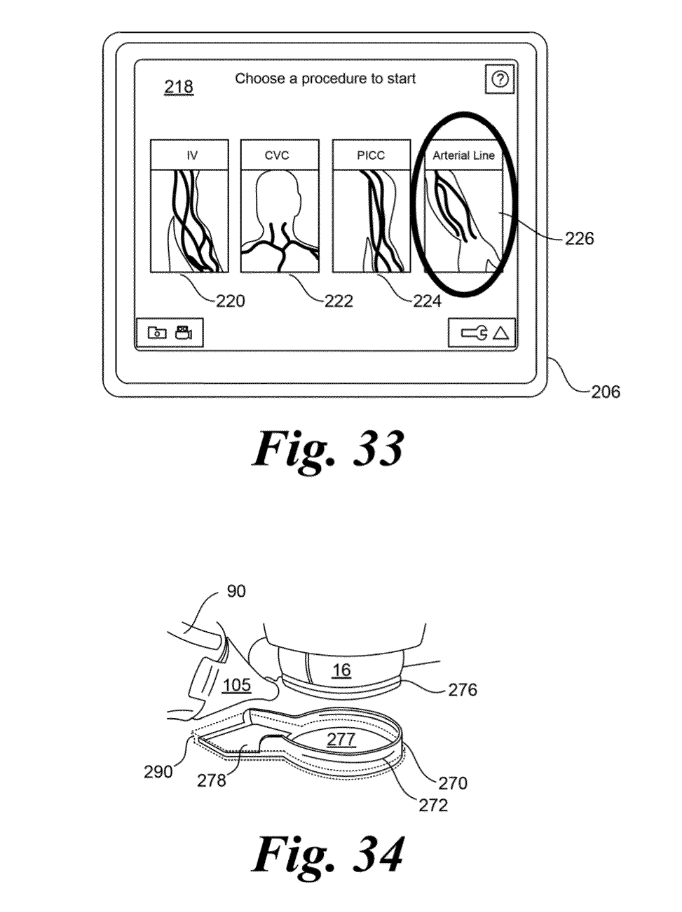

FIG. 33 schematically depicts the touch screen monitor 206 presenting a return to the home screen 218 illustrating that the arterial line procedure 226 has been touch screen selected by the user as indicated by the oval. The arterial line procedure 226 brings up menu items to conduct this blood vessel access procedure. Likewise, but not shown in FIG. 33, the other icons call up related procedures.

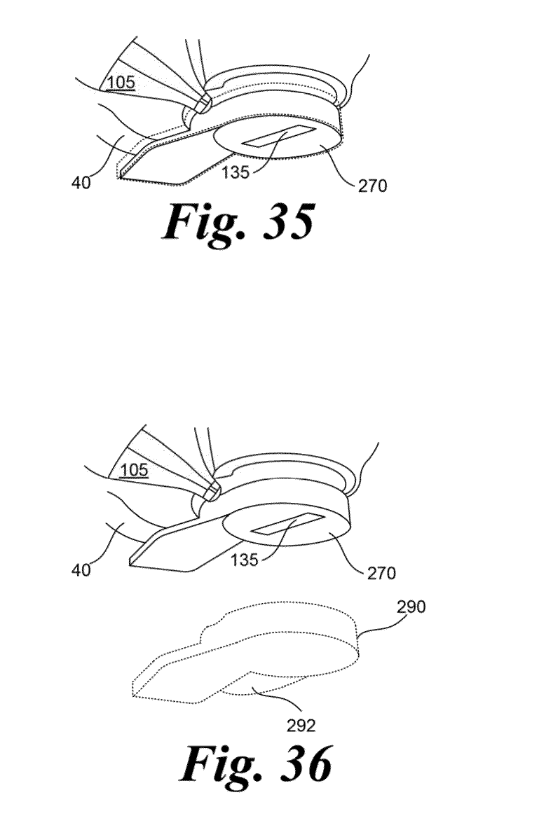

FIGS. 34-36 schematically depict attaching a sterile transducer cap 270 to transducer support 16 and adjacent friction hinge region of the injector arm 40. FIG. 34 illustrates that the internal walls of the cap 270 have ridges 272 that are configured to snap fit into the groove 276 of transducer support 16 to hold the sterile cap against the bottom surface of the transducer base 276. Around the cap 272 is a complementary shaped cap package cover 290 (shown in dotted lines). On the inside is a transparent membrane 277 of the cap 270 to which sonic coupling gel may be applied so that upon pressing and snap fitting the cap 270 against the bottom of the support 16 and engagement of the ridges 272 with groove 276, the cap 270 is held against the transducer base 16 such that the sonic gel will spread out and cover over the rotatable transducer 135. Adjacent to the transparent membrane 277 is arm cover 278 that covers the outer housing near the friction hinge 41 end of the injector arm 40 to prevent the end of injector arm 40 from contacting the patient's skin. (FIG. 35). Thereafter, as shown in FIG. 36, the complimentary shaped cap packaging cover 290 may be grasped by extension 292 to peel away the cover 290 from the snapped-in-place cap 270 that remains on the face of transducer base 16 of device 10.

FIGS. 37-39 schematically depict the covering of the handset 10 with a sterile sheath 300 and attachment of the needle-and-cannula cartridge 90 to a sheath enveloped handset 10 via fittings attached to the surface of the sheath. FIG. 37 depicts overlaying an unrolled sheath 300 to the handset 10 wherein the injector arm 40 is rotated to be vertically aligned with the transceiver 12. Accompanying the sheath 300 are fittings including a pair of injector adapters 302 each having a platform extension 306. The injector adapters 302 press into the slots of injector arm's 40 moveable platforms 50 and 52 and are held in place. FIG. 38 depicts the platform extensions 306 outwardly deployed to engage with the cassette's 90 slideable mounts 92/98 (not shown) while in the home position. FIG. 39 depicts the attachment of the cassette 90 to the sheath enveloped injector arm 40 through the connectors 56 and 58 (not shown) and slideable mounts 92/98 (not shown).

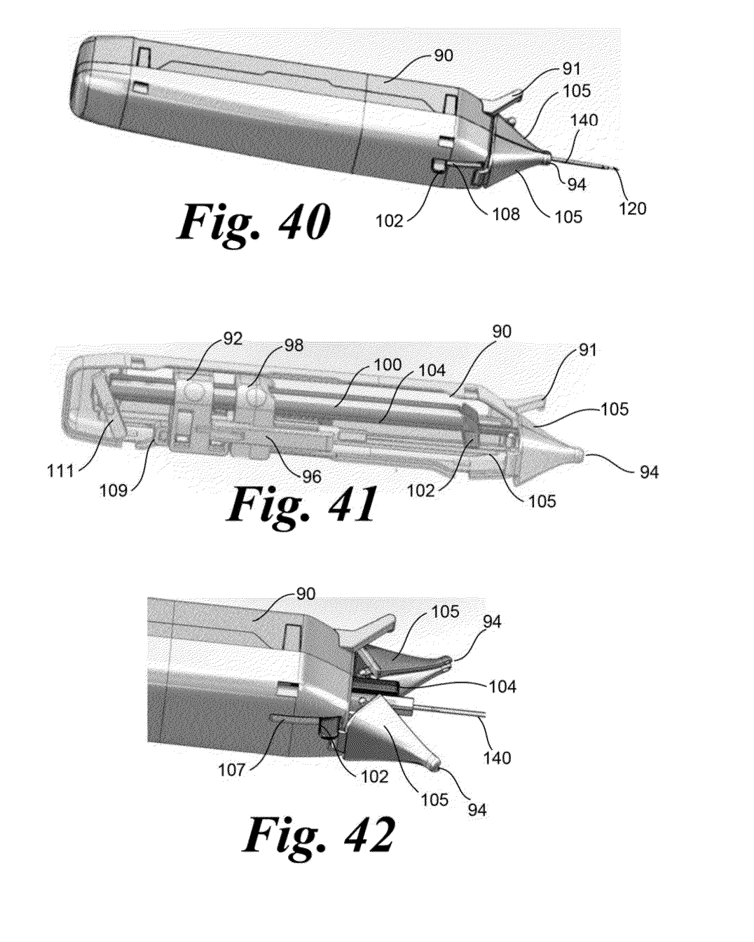

FIG. 40 depicts a perspective view of the cartridge 90. Cannula release 102 is shown in a rearward position in slot 108. Cannula 140 overlaps needle 120 and are shown projecting from the aperture of the needle/cannula guide 94 that is created by the closed swing doors 105.

FIG. 41 schematically depicts a partial cut-away and perspective view of the cartridge 90. The cannula release 102 is connected with a release bar 100 that is slidably engageable through aligned apertures of the slideable mounts 92/98. Extending from the release bar 100 is ramming bar 104 that is engageable with the swing doors 105.

Alternatively, the ramming bar 104 may be made to engage swing doors 105 when the rearward protrusion 109 of slideable needle mount 92 engages against rotatable lever 111 that pivots and causes the release bar 100 to move in the direction of the swing doors 105.

FIG. 42 schematically depicts a close-up of the cannula release 102 that pushes open swing guide doors 105 upon being slid forward within slot 107. The swung open door 105 illustrates the half apertures of the full aperture the needle guide 94 assumes when the swinging doors 105 are closed. Stated differently, each swinging door 105 possesses half the aperture or half of the needle guide 94. Forward motion of the cannula release 102 towards the guide aperture 94 causes the ramming bar 104 to press against the rearward lips of the swing doors 105 and thus push doors 105 open to create a space larger than the space defined by the needle guide aperture 94 when the doors are closed.

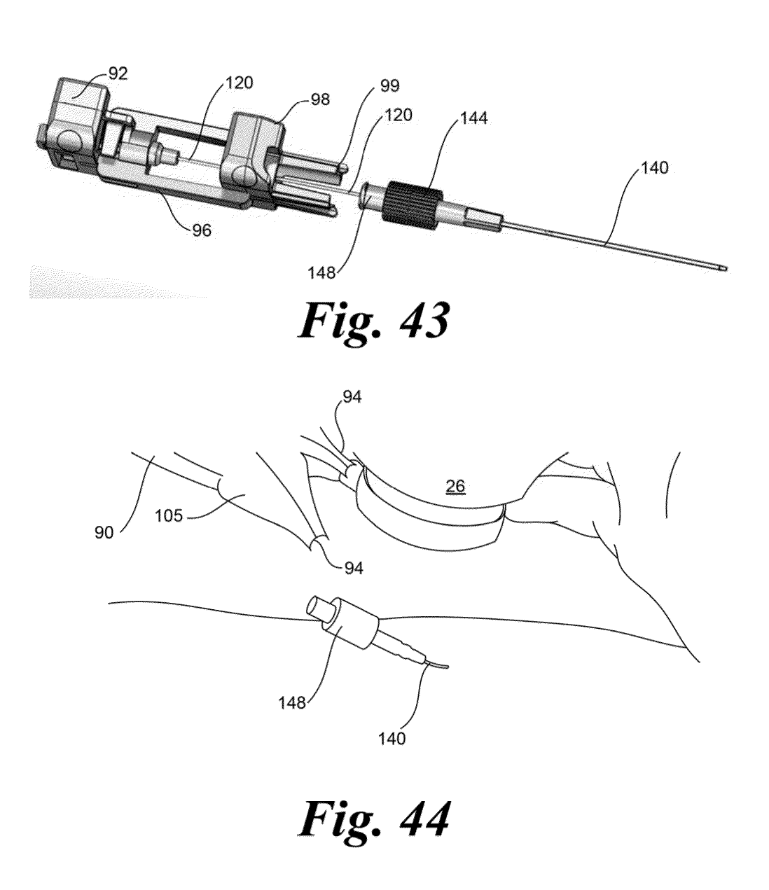

FIG. 43 schematically depicts needle slideable mount 92 retraction from slideable cannula mount 98 and cannula mount's 98 pinch holder 99 is allowed to relax and expand away from the hub 148 attached to cannula check valve 144. The hub 148 is configured to detachably attach with syringe and other fittings used in intravenous fluid or drug delivery or blood withdrawing procedures. The check valve 144 is has a puncturable septum (not shown) that is configured to reseal upon retraction of the needle 120 from the cannula 140. As the needle mount 92 retracts away from the cannula mount 98, the clamping action of side bars 96 is gradually reduced while they are gradually drawn away from the cannula mount 98, until there is no clamping action upon complete disengagement of the slide bars 96 from the slideable cannula mount 98. Release of the external portion of the cannula 140, that is the hub 148 and attached check valve 144, is made possible by the de-grasping action of the knurled end fingers of the now-relaxed pinch holder 99 that spread apart upon retraction of the side bars 96 and no longer pinch hold the distal edge of the check valve 144, thereby allowing the hub 148 and check valve 144 to move clear of the newly created wider space made possible by the opening of swing doors 105 as a consequence of the ramming action of ramming bar 104 conveyed to the proximal edges of the swing doors 105.

FIG. 44 schematically depicts removal of the handset 10 with opened guide doors 105 from the external portion of cannula 140 wherein the hub 148 and check valve 144 is seen extending outside the patient's arm with the internal portion of cannula 140 left in place residing inside the blood vessel lumen of the patient' arm. Inside the check valve 144 is a septum (not shown). The septum is configured to be pierced by the needle 120 and provide low friction back and forth slideability or movement of the needle 120 as a consequence of the back and forth movement of the slideable needle mount 92. The back and forth movement of the needle 120 through the septum occurs without imposing significant pushing or pulling forces onto the septum as a consequence of the low friction material comprising the septum. Thus there is no significant tugging or pushing forces conveyed to the hub 148 or check valve 144 by the slideable cannula mount 98 and as a result the positioning of the catheter or cannula 140 within the blood vessel is left undisturbed when the needle 120 is withdrawn. The septum is also configured with materials designed to sufficiently re-seal or close when the needle 120 is removed from the septum to prevent back flushing or escape of blood fluids from the hub 148.

With further regards to FIGS. 40-44, the swinging doors 105 are swung open by three mechanisms. First, an opening action may be engaged manually by the user who slides or pushes the cannula release 102 forward towards the patient within slot 108, thus causing the ramming action of ramming bar 104. Second, by causing mechanized forwardly-directed movement towards the patient of the slideable cannula mount 98 by signaling the moveable platform 52 to move forward towards the patient upon the user pressing the forward pushbutton 44 of controller 47. This causes the forward motion of release bar 100 and its ramming bar 104 extension against the rear portions of the swinging doors 105. Third, by mechanized rearward movement of the slideable needle mount 92 conveyed by moveable platform 50 via the user pressing the rearward pushbutton 42 of controller 47. This causes the needle mount's 92 rearward extension 109 to mechanically push the lower portion of the rotatable lever 111 to pivot such that the upper portion of the rotatable lever 111 mechanically engages the forward motion of release bar 100 and its ramming bar 104 extension towards the patient and against the rear portions of the swinging doors 105.

While the preferred embodiment of the invention has been illustrated and described, as noted above, many changes can be made without departing from the spirit and scope of the invention. For example, in alternate embodiments the display screens 206 may include the sections to display voice recorded alphanumeric messages during blood vessel access procedures 220-226.

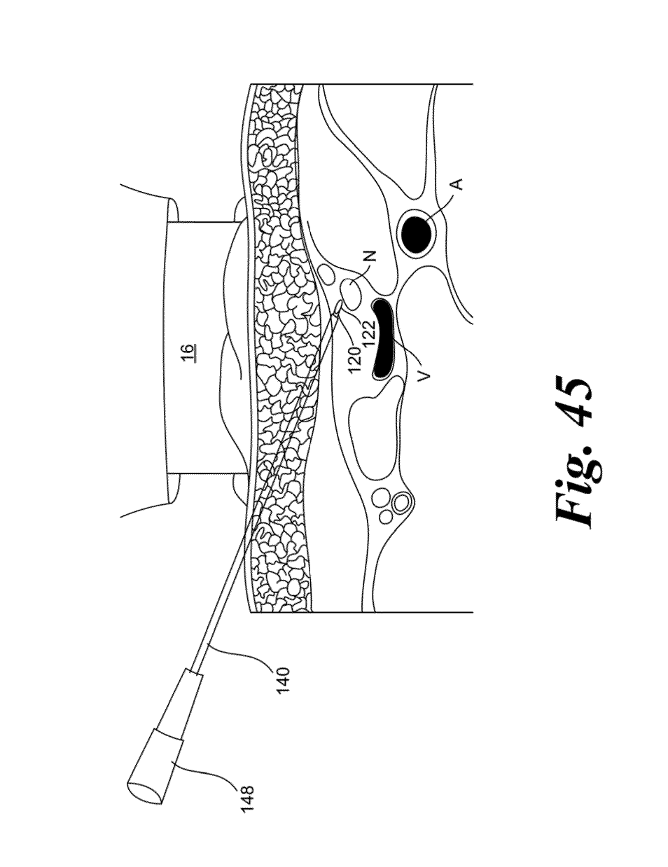

FIG. 45 schematically illustrates placement of the needle 120 and overlapping cannula 140 adjacent to a nerve within an insonified region of the patient's neuro-vasculature. The transducer base 16 is shown held against the patient's skin. Here the cannula 140 and the bevel 122 of the needle 120 is shown placed adjacent to a nerve N within the patient's vasculature. The external portion of the cannula 140, here represented by the hub 148, is available for syringe attachment and injection of nerve blocking solutions to the nerve N. Adjacent to the nerve N within the patient's neuro-vasculature is a compressed vein V and substantially un-compressed artery A. As with viewing blood vessels in short axis or long axis cross-sectional view, the process of placing the bevel 122 of the needle 120 nearby a nerve N, and the process of removing the needle 120 from the cannula 140 left adjacent to the nerve N, can be similarly viewed in short and long-axis cross sectional views.

With regards to FIG. 45, the neuro-vascular access system operated by a user for accessing a nerve within the patient's vasculature utilizes the ultrasound transceiver 12 that is configured for left-handed or right-handed holding, the ultrasound transceiver having the rotatable ultrasound transducer 135 that is in communication with the computer processing unit 202 connected with the monitor 206. The ultrasound transceiver 12 is handheld by the user against the patient to obtain rotatable views of an insonified region of the patient's neuro-vasculature relating to the signals of ultrasound echoes processed according to instructions executable by the computer processing unit. In pivotal connection with the ultrasound transceiver 12, the needle injector 40 utilizes motorized platforms 50 and 52 and the controller 47 operable by the user to change the position of the motorized platforms 50 and 52 and the rotatable transducer 135. The motorized platforms 50 and 52 engage with the cartridge's 90 first slideable mount 92 and the second slideable mount 98, and via position sensors located within the injector arm 40, positional information relative to the ultrasound transducer 135 is determined by the central processing unit 202 to generate a an overlay. The overlay provides the sighting and predicted trajectory the needle 120 and cannula 140 undertake when penetrating and travelling within the patient's neuro-vasculature as presented on the monitor's 206 ultrasound-based images.

In another alternate embodiment, the needle 120 may be held by the slideable cannula mount 98 and the slideable needle mount be configured to hold a shaft that is in slideable communication with the lumen of the needle 120. The neuro-vascular access system can then be deployed to do biopsy sampling wherein successive punches of the needle 120 can hold successive incremental samples by withdrawing the shaft at different depths as the needle 120 is plunged deeper into the neuro-vascular tissue via operation of the injector arm's 40 controller 47. The tissue biopsy sampling may furthermore be done via the access device 10 to bladders, uterus, lung, and other patient organs. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A neuro-vascular access system operated by a user comprising:
an ultrasound transceiver configured for left-handed or right-handed holding by the user against the surface of a patient, the ultrasound transceiver having a rotatable ultrasound transducer configured to obtain rotatable views of an insonified region, the rotatable transducer in communication with a computer processing unit, the ultrasound transceiver handheld by the user against the patient to obtain rotatable views of an insonified region of the patient's neuro-vasculature relating to the signals of ultrasound echoes processed according to instructions executable by the computer processing unit;
a needle injector pivotably attached to the ultrasound transceiver, the needle injector having at least one motorized platform and a controller operable by the user to rotate the rotatable transducer and to change the position of the at least one motorized platforms;
a cartridge configured for detachable connection with the injector and having at least one slideable mount configured for detachable connection with the at least one motorized platform, the at least one slideable mount including a needle configured for slideable motion between the cartridge and the patient's neuro-vasculature; and
a monitor configured to present images of the rotatable views.

2. The neuro-vascular access system of claim 1, wherein the cartridge includes a cannula configured for detachable connection with the at least one slideable mount and in slideable communication with the cartridge and the needle, and controllable by the controller to at least move within the cartridge, from the cartridge to the insonified region, and within the insonified region.

3. The neuro-vascular access system of claim 2, wherein the images are viewable and adjustable by the user operating the controller to obtain an orientation selected by the user to undertake positioning of the needle and the cannula adjacent to a nerve appearing within the insonified region.

4. The neuro-vascular access system of claim 2, wherein the motorized platforms include a first slideable mount and a second slideable mount, the first slideable mount in detachable connection with the needle and the second slideable mount in detachable connection with the cannula.

5. The neuro-vascular access system of claim 2, wherein the controller is configured to advance the first slideable mount synchronously with the second slideable mount towards or away from the patient's vasculature.

6. The neuro-vascular access system of claim 2, wherein the controller is configured to advance the first slideable mount towards or away from the patient's vasculature independently of the position of the second slideable mount.

7. The neuro-vascular access system of claim 2, wherein the controller is configured to advance the second slideable mount towards or away from the patient's vasculature independently of the position of the first slideable mount.

8. A neuro-vascular access system operated by a user comprising:
an ultrasound transceiver configured for left-handed or right-handed holding, the ultrasound transceiver having a rotatable ultrasound transducer in communication with a computer processing unit, the ultrasound transceiver handheld by the user against the patient to obtain rotatable views of an insonified region of the patient's neuro-vasculature relating to the signals of ultrasound echoes processed according to instructions executable by the computer processing unit;
a needle injector having motorized platforms and a controller operable by the user to change the position of the motorized platforms and the rotatable transducer, the motorized platforms including a first slideable mount and a second slideable mount, the needle injector being pivotally attached to the ultrasound transceiver and further configured to convey positional information relative to the ultrasound transceiver to the central processing unit;
a cartridge configured for detachable connection with the needle injector, the cartridge having a needle detachably attachable with the first slideable mount and a cannula detachably attachable with the second slideable mount, the needle having slideable connection within and disconnection from the lumen of the cannula, a cannula release, and a needle catch; and
a monitor configured to present images of the rotatable views, the images viewable and adjustable by the user operating the controller to obtain an orientation selected by the user to undertake placement of the needle and the cannula adjacent to a nerve appearing in the images according to positional information contained in an overlay having a trajectory overlaid on the images based on the positional information determined by the central processing unit, to retract the needle cannula placed adjacently to the nerve, and to secure the needle within the cartridge via the needle catch while keeping the interior end of the cannula residing adjacently to the nerve.

9. The neuro-vascular access system of claim 8, wherein the controller is configured to obtain rotatable views that include a substantially short axis cross-sectional view and a substantially long axis cross-sectional view of the nerve appearing within the insonified region.

10. The neuro-vascular access system of claim 9, wherein the substantially short axis cross-sectional view is employed by the user to align and monitor the needle for placement adjacent to the nerve and the substantially long axis cross-sectional view for monitoring the progress of needle retraction from cannula placed adjacently to the nerve.

11. The neuro-vascular access system of claim 8, wherein the controller is configured to move the first slideable mount synchronously with the second slideable mount towards or away from the patient's neuro-vasculature.

12. The neuro-vascular access system of claim 8, wherein the controller is configured to move the first slideable mount towards or away from the blood vessel independently from the second slideable mount and the second slideable mount towards or away from the patient's neuro-vasculature independently from the first slideable mount.

13. The neuro-vascular access system of claim 8, wherein the pivotally attached needle injector includes a friction hinge having position sensors configured to provide angular information of the friction hinge for determination of the trajectory for placing the needle adjacently to the nerve appearing within the insonified region.

14. The neuro-vascular access system of claim 8, wherein an angular change of the pivotally attached needle injector includes a friction hinge having position sensors configured to provide changes in the angular information undertaken by a change in the friction hinge position for determination of a change in trajectory of the cannula advanced beyond the needle residing adjacently to the nerve viewed from the images of the insonified region.

15. The neuro-vascular access system of claim 8, wherein the cannula release includes a pair of doors having an orifice sized to allow the passing of the cannula overlapping the needle without substantial sideway slippage while engaging needle and cannula placement whereupon engaging the cannula release swings open the doors to create a space sufficient to allow the cassette to be removed from the external portion of the cannula protruding above the patient's skin without displacing the internal portion of the cannula residing adjacently next to the nerve.

16. The neuro-vascular access system of claim 8, wherein the cannula includes a check valve having a septum configured to reseal sufficiently upon retraction of the needle from the cannula to prevent fluid leakage beyond the check valve.

17. The neuro-vascular access system of claim 8, wherein the base of the rotatable transducer may be covered by a sterilized cap for neuro-vascular access procedures requiring an aseptic arena.

18. The neuro-vascular access system of claim 8, wherein the transducer and the injector may be overlapped by a flexible sterile sheath for nuero-vascular access procedures requiring a sterile arena.
